(19) United States
(12) Patent Application Publication
(10) Pub. No.: US 2007/0196494 A1
(43) Pub. Date: Aug. 23, 2007

(54) LOW-FRIABILITY, PATIENT-FRIENDLY ORALLY DISINTEGRATING FORMULATIONS

(76) Inventors: Arnaud Grenier, Steinbrunn-Le-Haut (FR); Celine Decaudin, Saint Louis (FR); Dario Norberto Carrara, Oberwill (CH); Ubaldo Conte, Busto Arsizio (IT); Lauretta Maggi, Pavia (IT)

Correspondence Address: WINSTON & STRAWN LLP;PATENT DEPARTMENT, 1700 K STREET, N.W., WASHINGTON, DC, 20006, US

(21) Appl. No.: 11/670,293

(22) Filed: Feb. 1, 2007

Related U.S. Application Data
(60) Provisional application No. 60/774,228, filed on Feb. 17, 2006.

(51) Int. Cl. A61K 31/78

(52) U.S. Cl. 424/487

(57) ABSTRACT

The present invention relates to a rapidly disintegrating orally administratable solid dosage formulation that includes at least one active ingredient, at least one first disintegration agent that is at least one type-C methacrylic acid copolymer according to the U.S. Pharmacopoeia National Formulary US/NF, a second disintegration agent of crospovidone or a cross-linked povidone polymer derivative thereof, and a non-cariogenic diluent that does not increase glucose blood levels. The at least one first disintegration agent does not function as an enteric coating, insulation coating intended to protect active ingredient(s), or coating intended to mask taste or smell. The solid dosage form has a mass of about 50 to about 1000 mg, and the at least one first disintegration agent is present in the dosage form in an amount not exceeding 15%, with respect to the total weight of the dosage form. The second disintegration agent is present in the dosage form in an amount not exceeding 15% with respect to the total weight of the dosage form. The first and the second disintegration agent are present in total amounts that provide a weight ratio of about 1:1 to about 1:3, wherein the dosage form provides at least one of the in vitro or in vivo disintegration time that is less than 30 seconds, and has a friability of 1% or less according to the U.S. Pharmacopoeia test.

### CROSS-REFERENCES TO RELATED APPLICATIONS

This application claims the benefit of provisional application 60/774,228 filed on Feb. 17, 2006, the entire content of which is expressly incorporated herein by reference thereto.

### FIELD OF THE INVENTION

The present invention provides for orally disintegrating dosage forms having enhanced physical stability.

### BACKGROUND OF THE INVENTION

The present invention generally relates to orally disintegrating dosage forms. It relates in particular to orally disintegrating dosage forms whose manufacturing and packaging process are simple. It further relates to orally disintegrating dosage forms whose composition is patient-friendly since it does not contain excipients known to be responsible for tooth decay and/or dyspepsia and/or digestion intolerance reactions and/or modification of glycemia, among others.

The oral route is the predominant route for drug administration to children and seniors. The advantages and issues of solid drug formulations are very similar in the pediatric and geriatric sub-populations [M. Danish and M. K. Kottke, “Pediatric and Geriatric aspects of pharmaceutics, G. S: Banker, C. T. Rhodes (des.), Modern Pharmaceutics, 3^{rd} Edition, pp. 809-842, Marcel Dekker, New York 1996]. 43.2% of parents reports difficulties in giving per oral medications to their children, mainly due to difficulties when swallowing tablets [G. K. Steffensenm A. Pachai, S. E. Pedersen, “Peroral medicinsk behandling af born—er der problemer?”, Ugeskr. Laeger. 160:2249-2252, 1998]. Similarly, elderly patients also encounter difficulties with commercially available oral drug formulations, and often ultimately crush them prior to administration [G. Stieve, “Der geriatrische Patient—Hinsehen, handeln, helfen”, Pharm. Ztg. 145: 3413-3419, 2000]. Orodispersible drug formulations, that are intended to rapidly dissolve in the buccal cavity, are novel interesting approaches for pediatric and geriatric purposes.

Disintegrating agents most generally used for the production of orally disintegrating dosage forms (“Handbook of Pharmaceutical Excipients”, Fourth Edition, Pharmaceutical Press, 2003) are mostly of one of the two following types:
Insoluble, swelling agents: such as alginic acid and alginates, sodium carboxymethyl cellulose, chitosan, guar gum, magnesium aluminum silicate, methylcellulose, starch and derivatives thereof such as pregelatinized starch or sodium starch glycolate, which swell more or less in contact with saliva. All the aforementioned insoluble-type disintegrant agents are also known to be viscosity-increasing agents. It is therefore likely to reduce the amount of these insoluble ingredients in an orally disintegrating tablet formulation because they would form gels or slurries which may have an unpleasant mouth feel caused by the relatively high and heterogeneous viscosity.
Insoluble, non-swelling agents, which form colloidal dispersions with saliva may also be used as disintegrants in tablet applications, but one main drawback is that they impart a chalky, pasty unpleasant mouth feel when placed in the mouth. Such excipients comprise calcium phosphates and derivatives thereof, powdered cellulose, microcrystalline cellulose, and colloidal silicon dioxide, for instance.

Crospovidone, a water-insoluble synthetic cross-linked homo-polymer of N-vinyl-2-pyrrolidone, may be considered as intermediate between these two “categories” since although a water-insoluble polymer, it rapidly exhibits high capillary activity and pronounced hydration capacity, with however very little tendency to form gel which blocks the tablet pores and prevents further penetration of water into the deeper zones. In contrast, the swelling of crospovidone is limited, but still maintains a particle structure with a defined surface. Crospovidone is a so-called “wicking” agent that pulls water into pores, hence reducing the physical bonding forces between particles.

Ideally, a water-soluble disintegrant would be preferred in orally disintegrating tablets since it would readily dissolve in the saliva and therefore would not affect the mouth feel. Such a water-soluble disintegrant is disclosed in U.S. Pat. No. 6,696,085, the entire content of which is herein incorporated by reference, which is directed to the use of type-C polymethacrylate according to the U.S. Pharmacopoeia National Formulary US/NF as a disintegration agent in orally disintegrating tablets.

Furthermore, due to the hygroscopic nature of the ingredients used in orally disintegrating dosage forms necessary to achieve rapid disintegration when placed in the mouth, such formulations are often very sensitive to moisture and are weak. Therefore special conditions for handling, storage and packaging are often required. See U.S. Pat. No. 6,221,392, column 1, line 43-67 and column 2, line 1-14. In this aspect, U.S. Pat. No. 6,696,085 recites examples with a certain type-C polymethacrylate ingredient (hereinafter referred as “EUDRAGIT”) which friability “is comprised approximately between 0 and 25%”. However, all examples displayed a friability comprised between 0.9% and 7.4% or even more, as recited in claims 5 and 14, and friability values of the “EUDRAGIT” formulas always exceeded either friability values obtained for the “control” formulas, as in examples 2, 3 and 4, or 7%, as in examples 1 and 5 (see table herein).

| | Ex- | | | | |
|---|---|---|---|---|---|
| | ample 1 | Example 2 | Example 3 | Example 4 | Example 5 |
| “Control” | No data | 0.3% | 0.5% | 0.55% | No data |
| formula | | | | | |
| “EUDRAGIT” | 8.8% | 2.7% | 0.9% | 1.17% | 7.4% |
| formula | | | | | |

Furthermore, friability data presented in U.S. Pat. No. 6,696,085 has been generated using the abrasion drum test, also known as the “Roche abrasion test”. Based on an original design by Roche, the abrasion drum for carrying out tests into attrition comprises of a 20-cm diameter drum (see figure herein) with a series of baffles which carry the tablets to a predetermined height before sliding off and reproduces the action of the tablets rubbing against each other during transport.

However, the abrasion test is known to be less “severe” than the standard friability test described in the U.S. Pharmacopoeia National Formulary US 29/NF 24, because the drum in use in the friability test has a single curved baffle which allows the tablets to be tested to rise and then drop through a distance of approximately 156 mm (see figure below), which is much higher than in the abrasion test.

The present invention now provides an orally disintegrating dosage form having a friability value lower than the control formulation, said friability value fulfilling requirements of the United States Pharmacopoeia for tablet friability, i.e. “a maximum weight loss of not more than 1% of the weight of the tablets” (please see USP 29-NF 24, paragraph <1216>“TABLET FRIABILITY”, page 3046-3047). Please note that this specification is applicable to “conventional” tablets, i.e. not intended to dissolve and/or to disintegrate within the buccal cavity. In general, a maximum weight loss of not more than 0.8% to 1.0% is acceptable for most tablets. USP further states that “effervescent tablets and chewable tablets may have different specifications as far as friability is concerned, and these tablets normally require special packaging.” These dosage forms are intended to be dissolved or disintegrated prior to or upon administration.

U.S. Pat. No. 6,696,085 discloses examples containing dextrose as the preferred diluent (about 50% by weight of the total tablet). Examples 2, 3, 4 and 5 of U.S. Pat. No. 6,696,085 further contain a small amount of sorbitol (about 3.00% by weight of the total tablet) in order to “improve the compressibility of the mixture of powders”.

Sugars such as dextrose, sucrose, glucose, saccharose, etc. . . . , are well known to cause tooth decay (cariogenic action), to increase calories intake, and to influence glycemia levels. Replacement of sugars in food (i.e. promoting tooth decay) is considered as very important by Health Agencies and Dentists Associations worldwide (please see “Sugar Substitutes: Americans Opt for Sweetness and Lite”, by John Henkel, in FDA Consumer magazine, November-December 1999). This trend initiated in the Food Industry has also gained lately the Pharmaceutical Industry, as witnessed by the approval given on September 2005 by the FDA to market in the US a new sugar-free formulation of an oral transmucosal fentanyl citrate “lollipop” bioequivalent to the original formulation containing hydrated dextrates and confectioner's sugar.

Simple sugars, such as fructose, lactose, maltodextrin, isomalt, etc., affect the blood glucose levels less dramatically than regular table sugar. In addition sugar alcohols, such as sorbitol, xylitol, lactitol, mannitol, maltitol, galactitol, erythritol, inositol, ribitol, dithioerythritol, dithiothreitol, and glycerol do not contribute to dental caries (cavities) since bacteria living in the mouth are unable to use them as a source of energy. Please see “Sugar Substitutes: Americans Opt for Sweetness and Lite” referenced herein above.

Lactose however can cause abdominal discomfort and bloating in lactose-intolerant people, i.e. in people suffering from a lack or shortage of the enzyme called lactase responsible for breaking down of lactose into glucose and galactose in the small intestine. Hence left unabsorbed by the body, the perfect conditions found in the intestines help the lactose to ferment and this leads to the formation of gases. A particular gas is methane that is usually the cause for the pain and aggressive flatulence. Common symptoms of lactose intolerance caused by the fermentation of lactose include nausea, cramps, bloating gas, wind, diarrhea, which may begin from after half an hour to 2 hours after eating or drinking foods containing lactose. Persons who suffer from lactose deficiency and do not avoid lactose may suffer from weight loss and malnutrition. The severity of symptoms varies depending on the amount of lactose each individual can tolerate. Lactose intolerance is a very common disorder, very often ignored, that is present in many people, especially in babies. Between 30 and 50 million Americans are lactose intolerant. Certain ethnic and racial populations are more widely affected than others. As many as 75 percent of all African Americans and American Indians and 90 percent of Asian Americans are lactose intolerant. The condition is least common among persons of northern European descent (please see “Lactose Intolerance” web page, edited by the National Digestive Diseases Information Clearinghouse, NIH Publication No. 03-2751, March 2003, http://digestive.niddk.nih.gov/ddiseases/pubs/lactoseintolerance). Similar intolerance reactions also exist for other simple sugars and sugar alcohols, such as for fructose and sorbitol, but their occurrence and the severity of the symptoms is much lower.

EMEA Guideline on “Excipients in the label and package leaflet of medicinal products for human use”, updated July 2003 (Medicinal products for human use; Safety, environment and information, VOLUME 3B, Document # CPMP/463/00, the entire content of which is incorporated herein as reference), is a Commission guideline that contains warning statements relating to the presence in medicinal products of certain excipients known to have a recognized action or effect, and provides a list of information on those excipients, knowledge of which is important for the safe and effective use of the medicinal product.

As shown below by extracts of the Annex listing such excipients requiring special labeling, fructose-, galactose-, glucose-, maltitol-, invert sugar-, lactitol-, lactose-, sorbitol-, sucrose-, and wheat starch-containing medicines should not be taken by patients with rare hereditary problems of intolerance and/or particular enzyme deficiencies/insufficiencies. Mannitol and xylitol are the only direct-compression diluents commonly used in the Pharmaceutical Industry that do not present such a “Zero” threshold.

| | | | | |
|---|---|---|---|---|
| Fructose | Oral | Zero | If you have been told by your | SPC Proposal: |
| | Parenteral | | doctor that you have intolerance | Patients with rare |
| | | | to some sugars, contact your | hereditary problems |
| | | | doctor before taking this | of fructose |
| | | | medicinal product. | intolerance should |
| | | | | not take this |
| | | | | medicine. |
| | | 5 g | Contains x g fructose per dose. | |
| | | | This should be taken into account | |
| | | | in patients with diabetes mellitus. | |
| | Oral | Zero | Maybe harmful to the teeth. | Information to be |
| | liquids, | | | included only when |
| | lozenges, | | | the medicinal |
| | and | | | product may be |
| | chewable | | | intended for chronic |
| | tablets | | | use, e.g. for two |
| | | | | weeks or more |
| Galactose | Parenteral | Zero | If you have been told by your | SPC Proposal: |
| | | | doctor that you have intolerance | Patients with rare |
| | | | to some sugars, contact your | hereditary problems |
| | | | doctor before taking this | of galactose |
| | | | medicinal product | intolerance, e.g. |
| | | | | galactoaemia, |
| | | | | should not take this |
| | | | | medicine |
| | Oral | Zero | If you have been told by your | SPC Proposal: |
| | | | doctor that you have intolerance | Patients with rare |
| | | | to some sugars, contact your | hereditary problems |
| | | | doctor before taking this | of galactose |
| | | | medicinal product | intolerance, e.g. |
| | | | | galactoaemia, or |
| | | | | glucose-galactose |
| | | | | malabsorption, |
| | | | | should not take this |
| | | | | medicine |
| | Oral | 5 g | Contains x g galactose per dose. | |
| | Parenteral | | This should be taken into account | |
| | | | in patients with diabetes mellitus. | |
| Glucose | Oral | Zero | If you have been told by your | SPC Proposal: |
| | | | doctor that you have intolerance | Patients with rare |
| | | | to some sugars, contact your | glucose-galactose |
| | | | doctor before taking this | malabsorption, |
| | | | medicinal product | should not take this |
| | | | | medicine |
| | Oral | 5 g | Contains x g glucose per dose. | |
| | Parenteral | | This should be taken into account | |
| | | | in patients with diabetes mellitus. | |
| | Oral | Zero | Maybe harmful to the teeth | Information to be |
| | liquids, | | | included only when |
| | lozenges, | | | the medicinal |
| | and | | | product may be |
| | chewable | | | intended for chronic |
| | tablets | | | use, e.g. for two |
| | | | | weeks or more |
| Hydrogenated | Oral | Zero | If you have been told by your | SPC Proposal: |
| Glucose syrup | | | doctor that you have intolerance | Patients with rare |
| (or Maltitol | | | to some sugars, contact your | hereditary problems |
| Liquid) | | | doctor before taking this | of fructose |
| | | | medicinal product | intolerance should |
| | | | | not take this |
| | | | | medicine. |
| | | 10 g | May have a mild laxative effect | |
| | | | Calorific value 2.3 kcal/g of | |
| | | | hydrogenated glucose syrup | |
| Invert sugar | Oral | Zero | If you have been told by your | SPC Proposal: |
| | | | doctor that you have intolerance | Patients with rare |
| | | | to some sugars, contact your | hereditary problems |
| | | | doctor before taking this | of fructose |
| | | | medicinal product | intolerance or |
| | | | | glucose-galactose |
| | | | | malabsorption |
| | | | | should not take this |
| | | | | medicine. |
| | | 5 g | Contains x g of a mixture of | |
| | | | fructose and glucose per dose | |
| | | | This should be taken into account | |
| | | | in patients with diabetes mellitus. | |
| | Oral | Zero | Maybe harmful to the teeth | Information to be |
| | liquids, | | | included only when |
| | lozenges, | | | the medicinal |
| | and | | | product may be |
| | chewable | | | intended for chronic |
| | tablets | | | use, e.g. for two |
| | | | | weeks or more |
| Lactitol, E966 | Oral | Zero | If you have been told by your | SPC Proposal: |
| | | | doctor that you have intolerance | Patients with rare |
| | | | to some sugars, contact your | hereditary problems |
| | | | doctor before taking this | of fructose |
| | | | medicinal product | intolerance, |
| | | | | galactose |
| | | | | intolerance, e.g. |
| | | | | galactoaemia, or |
| | | | | glucose-galactose |
| | | | | malabsorption, |
| | | | | should not take this |
| | | | | medicine |
| | | 10 g | May have a mild laxative effect | |
| | | | Calorific value 2.1 kcal/g lactitol | |
| Lactose | Oral | Zero | If you have been told by your | SPC Proposal: |
| | | | doctor that you have intolerance | Patients with rare |
| | | | to some sugars, contact your | hereditary problems |
| | | | doctor before taking this | of galactose |
| | | | medicinal product | intolerance, the |
| | | | | Lapp lactose |
| | | | | deficiency or |
| | | | | glucose-galactose |
| | | | | malabsorption, |
| | | | | should not take this |
| | | | | medicine. |
| | | 5 g | Contains x g lactose (x/2 g glucose | |
| | | | and x/2 g galactose) per dose. | |
| | | | This should be taken into account | |
| | | | in patients with diabetes mellitus. | |
| Maltitol E965 | Oral | Zero | If you have been told by your | SPC Proposal: |
| and | | | doctor that you have intolerance to | Patients with rare |
| Isomaltitol E953, | | | some sugars, contact your doctor | hereditary problems |
| Maltitol | | | before taking this medicinal | of fructose |
| Liquid | | | product | intolerance should |
| (see | | | | not take this |
| Hydrogenated | | | | medicine |
| Glucose Syrup) | | 10 g | May have a mild laxative effect | |
| | | | Calorific value 2.3 kcal/g maltitol | |
| | | | (or isomaltitol) | |
| Mannitol, E421 | Oral | 10 g | May have a mild laxative effect | |
| Sorbitol E420 | Oral | Zero | If you have been told by your | SPC Proposal: |
| | Parenteral | | doctor that you have intolerance | Patients with rare |
| | | | to some sugars, contact your | hereditary problems |
| | | | doctor before taking this | of fructose |
| | | | medicinal product | intolerance should |
| | | | | not take this |
| | | | | medicine |
| | Oral | 10 g | May have a mild laxative effect | |
| | | | Calorific value 2.6 kcal/g of | |
| | | | sorbitol | |
| Sucrose | Oral | Zero | If you have been told by your | SPC Proposal: |
| | | | doctor that you have intolerance to | Patients with rare |
| | | | some sugars, contact your doctor | hereditary problems |
| | | | before taking this medicinal | of fructose |
| | | | product | intolerance, |
| | | | | glucose-galactose |
| | | | | malabsorption or |
| | | | | sucrase-isomaltase |
| | | | | insufficiency should |
| | | | | not take this |
| | | | | medicine |
| | | 5 g | Contain: x g of sucrose per dose. | |
| | | | This should be taken into account | |
| | | | in patients with diabetes mellitus. | |
| | Oral | Zero | Maybe harmful to the teeth | Information to be |
| | liquids, | | | included only when |
| | lozenges, | | | the medicinal |
| | and | | | product may be |
| | chewable | | | intended for chronic |
| | tablets | | | use, e.g. for two |
| | | | | weeks or more |
| Wheat starch | Oral | Zero | Suitable for people with coeliac | Wheat starch may |
| | | | disease. Patients with wheat | contain gluten, but |
| | | | allergy (different from coeliac | only in trace |
| | | | disease) should not take this | amounts, and is |
| | | | medicine. | therefore |
| | | | | considered safe for |
| | | | | people with coeliac |
| | | | | disease. (Gluten in |
| | | | | wheat starch is |
| | | | | limited by the |
| | | | | test for total protein |
| | | | | described in the |
| | | | | PhEur monograph.) |
| Xylitol | Oral | 10 g | May have a laxative effect | |
| | | | Calorific value 2.4 kcal/g xylitol | |

The present invention also provides an orally disintegrating dosage form that is patient-friendly in the sense that it does contain neither complex sugars nor simple sugars responsible for increase of glycemia, for tooth decay, or for intestinal discomfort.

The patient-friendly aspect of the present invention may be further enhanced by the fact that compositions according to the present invention are substantially free of aspartame, which may be harmful for people with phenylketonury.

The patient-friendly aspect of the present invention is even further enhanced by the fact that compositions according to the present invention are substantially free of insoluble swelling disintegrant agents responsible for increase of viscosity or for chalky, pasty mouth feel.

Lehman, in “Formulation of Controlled Release Tablets with Acrylic Resins”, Acta Pharma. Fenn, 93(2), pp. 55-74, (1984), teaches the use of EUDRAGIT polymethacrylate polymer (A) as the sole disintegration agent in a coating for sustained release tablets (p. 57, and pp 63-64), (B) in a compressed mixture with theophylline by itself in an amount of 5-15% (p. 66) or (C) in a compressed mixture with theophylline with other disintegration agents such as microcrystalline cellulose, lactose, dextrose, and sucrose in a weight ratio of 1:3 (10:30) to 2:5 (10:25) to 1:2 (15:30) to 3:5 (15:25) (p. 67). Lehman further discloses dextrose, sucrose, lactose, and cellulose derivatives as excipients.

U.S. Pat. No. 5,409,711, to Mapelli et al., relates to a coating for orally administered drugs for masking the taste of such drugs. More particularly, example 1 of this patent discloses the use of EUDRAGIT L 100-55 in an amount (192 g) which is less than that of other disintegration agents, such as microcrystalline cellulose and KOLLIDON CL (412 g), i.e. in a type-C polymethacrylate:crospovidone ratio of 1:2.15 or lower. Mapelli et al. do not attribute any particular significance to the use of type-C polymethacrylate as a disintegrant agent, but instead disclose that type-C polymethacrylate is used as a coating agent. Mapelli et al. emphasize the use of type-C polymethacrylate as a coating agent by further specifying that any one of a wide variety of polymeric materials are suitable for preventing the person that is consuming the tablet from experiencing the poor taste of the active ingredient. This patent mentions that an acidic compound is mixed with the coated core for reducing or preventing the dissolution of the EUDRAGIT L100-55 membrane in the buccal cavity. In addition, oral tablets according to this patent result in a relatively slow disintegration rate.

U.S. Pat. No. 6,074,669 to Nagaprasad et al. discloses combinations of type-C polymethacrylate and a second disintegration agent, in relative amounts of 1:15 (Example 1) or 1:4 (Example 2 and 3), i.e. in relative amounts that are much higher than those that are presently claimed (i.e., from 1:1 to 1:3). Furthermore Nagaprasad teaches the use of Methocel K 100M and HPC-M (hydroxypropylmethylcellulose, or hypromellose) as a second disintegrating agent. Thus, the disintegration rate of Nagaprasad's formulations are also relatively slow.

U.S. Pat. No. 7,029,699, to Robinson et al. relates to soft, convex-shaped chewable tablets obtained by dry blending and direct pre-compression/compression, having a friability less than 1.00%. This patent recites formulations containing mannitol as the water-disintegrating, compressible carbohydrate and EUDRAGIT E 100. Mannitol is simply an example of a water-disintegrating, compressible carbohydrate of preferred choice, and others such as sorbitol, maltitol, dextrose, sucrose, xylitol, lactose, and mixtures thereof can be used. Furthermore, the mannitol quality, that is used is granular mannitol (see Example I, Example II, and Example III). EUDRAGIT E 100, though also a polymethacrylate, is not a type-C polymethacrylate and is used only as part of the polymer coating system necessary to overcome the bad taste of some active drugs.

The resulting monolithic formulations (e.g., chewable tablets) are difficult to administer to young children and elderly patients, who either physically cannot (due to the absence of teeth) or do not know how to crush tablets. Indeed, a very recently published reflection paper of the European Regulatory Office provided a matrix for the applicability of drug dosage forms for children aged 18 or less (see Table herein). The safety of chewable tablets for children of 2 years or less has not been proved, conversely to orally disintegrating formulations, which were found applicable in newborn infants and toddlers. See “Formulations of choice for the pediatric population, Committee for medicinal products for human use, EMEA/CHMP/PEG/194810/2005.

TABLE 1
Applicability of peroral formulation in children (according to EMEA/CHMP/PEG/194810/2005)

| | Preterm | | | | | |
|---|---|---|---|---|---|---|
| | newborn | Term | Infants and | | | |
| | infants | newborn | toddlers | Pre-school | School- | |
| Dosage form | (<0 d) | infants (0–28 d) | (1 m–2 y) | children (2–5 y) | children (6–11 y) | Adolescents |
| Solution/drops | applicable | good | best and | best and | preferred | preferred |
| | with | applicability | preferred | preferred | acceptability | acceptability |
| | problems | | applicability | applicability | | |
| Emulsion/ | applicable | probably | good | best and | preferred | Preferred |
| suspension | with | applicable | applicability | preferred | acceptability | acceptability |
| | problems | not | | applicability | | |
| | | preferred | | | | |
| Effervescent | applicable | good | best and | best and | preferred | preferred |
| formulation | with | applicability | preferred | preferred | acceptability | acceptability |
| | problems | | applicability | applicability | | |
| Powders/ | not | applicable | applicable | good | preferred | dosage form |
| multiparticulates | applicable | with | with | applicability | acceptability | of choice |
| | | problems | problems | | | |
| Tablets | not | not | not | probably | preferred | dosage form |
| | applicable | applicable | applicable | applicable | acceptability | of choice |
| | | | | not | | |
| | | | | preferred | | |
| Capsules | not | not | not | applicable | preferred | dosage form |
| | applicable | applicable | applicable | with | acceptability | of choice |
| | | | | problems | | |
| Orodispersable | not | applicable | probably | good | dosage form | dosage form |
| formulation | applicable | with | applicable | applicability | of choice | of choice |
| | | problems | not | | | |
| | | | preferred | | | |
| Chewable | not | not | not | probably | dosage form | dosage form |
| tablets | applicable | applicable | applicable | applicable | of choice | of choice |
| | | | | not | | |
| | | | | preferred | | |

U.S. Pat. No. 6,221,392, to Khankari et al. entitled “Rapidly dissolving robust dosage form,” is directed to hard, compressed, rapidly dissolvable dosage forms adapted for direct oral dosing comprising an active ingredient and a matrix including a non-direct compression filler and a lubricant. Dosage forms described in this patent are claimed to have a friability of about 2% or less when tested according to the U.S.P. The patentrelies, in particular, on the use of non-direct compression filler and unusual high levels of lubricant in an attempt to balance the various competing objectives of the orally disintegrating dosage forms, namely compressibility at conventional pressures, sufficient hardness and friability allowing for certain processing and packaging advantages, and rapid dissolution in the mouth. See column 8, lines 64-67 and column 9, lines 1-8. Rationale for selection of non-direct compression filler is later explained in column 9, lines 15-59. Particularly preferred fillers are non-direct compression sugars and sugar alcohols which have at least 85% of the particles significantly under 100 microns. Several examples of the patent disclose a formulation containing powdered mannitol as the non-direct compression filler: see Examples 4, 5, 6, 7 and 8.

A need therefore exists for oral dosage forms having improved taste, improved palatability, and not containing excipients responsible for disagreements in certain specific patient sub-populations, e.g. for instance digestion discomfort, tooth decay, increase of glycemia, but which exhibit low friability so that they may be processed with standard bulk handling equipment and packaged in conventional packaging solutions.

### SUMMARY OF THE INVENTION

The present invention relates to a robust, direct-compressed, rapidly disintegrating oral tablet for administration of pharmaceutically active ingredients. The tablet of the present invention includes at least one active ingredient and a matrix composed of a first water-soluble disintegrant of the type-C polymethacrylate type according to the U.S. Pharmacopoeia National Formulary US/NF, at least a second disintegration agent of crospovidone or a cross-linked povidone polymer derivative thereof, and a non-cariogenic diluent. The at least one first disintegration agent does not function as an enteric coating, insulation coating intended to protect active ingredient(s), or coating intended to mask taste or smell. The solid dosage form has a mass of about 50 to about 1000 mg, and the at least one first disintegration agent is present in the dosage form in an amount not exceeding 15% with respect to the total weight of the dosage form. The second disintegration agent is present in the dosage form in an amount not exceeding 15% with respect to the total weight of said dosage form. The first and the second disintegration agent are present in total amounts that provide a weight ratio of about 1:1 to about 1:3, wherein the dosage form provides at least one of the in vitro or in vivo disintegration time that is less than 30 seconds. The tablet is further characterized by a friability of 1.00% or less according to the U.S. Pharmacopoeia test.

It is desirable that the tablet of the present invention dissolves in about 30 seconds or less (depending on the tablet weight: generally, the higher the tablet weight, the longer the disintegration time) when put either in water or in simulated saliva fluid at a temperature comprised between 35° C. and 39° C. or in the patient's mouth.

In a particularly preferred formulation in accordance with the present invention, there is provided a robust, direct-compressed, rapidly disintegrating oral tablet consisting in at least one active ingredient and a matrix comprising a water-soluble disintegrant, at least one disintegrant which is insoluble but non-swelling in water, and a sugar alcohol. The tablet matrix comprises not more than 30% by weight of the total tablet of the disintegrants, the water-soluble disintegrant and the water-insoluble, non-swelling disintegrant being present in ratios ranging from 1:1 to 1:3, all being preferably freely to slightly soluble in water. The tablet matrix may further include flavors and flavor enhancers, sweeteners and lubricants, all preferably freely to slightly soluble in water.

The amount of insoluble excipients may represent not more than about 15% based on the total weight of the tablet matrix (i.e. active ingredient not included), therefore minimizing the potential grittiness experienced with orally disintegrating tablets.

In one embodiment, the non-cariogenic diluent consists substantially of a sugar alcohol, such as mannitol and is present in an amount of between 25% and 85% by weight of the formulation. In another embodiment, the formulation is substantially free of lactose or fructose responsible for intestinal discomfort in populations suffering from sugar intolerance or which is substantially free of precursors being metabolized in the human body into lactose or fructose.

Preferably, the formulation also includes one or more diluents or fillers, sweeteners, binders, flavors and flavor enhancers, buffers, preservatives, antioxidants, lubricants, bioadhesive agents, colorants, flow agents, plasticizers, film forming agents, coating agents, polishing agents, shining agents, permeation enhancers, or mixtures thereof. Advantageously, the sweetener is not contraindicated in populations suffering from phenylketonury.

In general, the type-C methacrylic acid copolymer is a compound having the following formula:

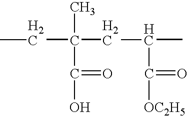

The crospovidone is typically the only insoluble swelling inactive ingredient present in the formulation. In a preferred embodiment, the hardness, friability and at least one of the in vitro or in vivo disintegration times of the dosage form is maintained over a storage period of at least six months at 40° C.

In another embodiment, the dosage forms of the present invention comprise an active ingredient useful to treat all kind of affections and diseases, such as, but not limited to, disorders and diseases affecting the gastrointestinal tract, the cardiovascular system, the central nervous system, the musculoskeletal system, the genitourinary system, the respiratory tract, the skin and the mucosa surfaces; endocrine disorders, diabetes, infections, nutrition disorders, allergic disorders, contraception, gynecological disorders, neoplastic disorders, addictive behaviors, pain, anesthesia and analgesia. In yet preferred embodiments, the active ingredient used to treat gastrointestinal tract is selected from the group consisting of antacids like calcium carbonate, H2-receptor antagonists like cimetidine, proton pump inhibitors like lansoprazole, cytoprotectants like misoprolol, laxatives like bisacodyl and antidiarrhoeals like loperamide; the active ingredient used to treat cardiovascular system is selected from the group consisting of: angiotensin II antagonists like candesartan, beta-blockers like carvedilol, calcium antagonists like amlodipine, potassium channel activators like nicorandil, diuretics like spironolactone, ACE inhibitors like enalapril, alpha-1 antagonists like prazosin or imidazoline, agonist like moxonidine, central alpha-agonists like clonidine, antiplatelet drugs like clopidogrel and hypolipidaemic agents like atorvastatin; the active ingredient used to treat central nervous system is selected from the group consisting of hypnotics like zolpidem, anxiolytics like buspirone, antipsychotics like clozapine, antidepressants and mood stabilizers like clomipramine, anti-emetics like granisetron, anticonvulsivants like lamotrigine, anti-Parkinson drugs like pramipexole, anti-Alzheimer drugs like donepezil, anti-ADHD like methylphenidate and narcoleptics like modafinil; the active ingredient used to treat pain is selected from the group consisting of analgesics and antipyretics like paracetamol and anti-migraine drugs like domperidone; the active ingredient used to treat musculoskeletal disorders is selected from the group consisting of non-steroidal anti-inflammatory drugs like meloxicam, anti-gout agents like allopurinol, muscle relaxants like dantrolene and neuromuscular drugs like distigmine; the active ingredient used to treat endocrine disorders is selected from the group consisting of hormones like testosterone, drugs for erectile dysfunction like sildenafil, corticosteroids like prednisolone, thyroid hormones like levothyroxine and drugs affecting bone metabolism like etidronate; the active ingredient used to treat diabetes is selected from the group consisting of sulfonylureas like glimepiride, meglitinides like repaglinide and thiazolidinediones like rosiglitazone; the active ingredient used to treat infections is selected from the group consisting of antibiotics like amoxicillin, antifungals like terbinafine, antituberculous drugs like rifampicin, anti-malaria like chloroquine, anthelmintics like mebendazole, anti-virals like acyclovir and immunosuppressants like tacrolimus; the active ingredient used to treat genito-urinary system is selected from the group consisting of overactive bladder drugs like tolterodine; the active ingredient used to treat nutrition disorders is selected from the group consisting of vitamins, electrolytes, antiobesity agents like sibutramine; the active ingredient used to treat respiratory system is selected from the group consisting of bronchodilatators like zafirlukast, expectorants, antitussives and decongestants like carbocisteine; the active ingredient used to treat allergic disorders is selected from the group consisting of anti-allergics like cetirizine; the active ingredient used to treat skin diseases is selected from the group consisting of: anti-acne like isotretinoin; the active ingredient used for contraception is selected from the group consisting of estrogens like ethinyl-estradiol and progestogens like norethindrone acetate; the active ingredient used to treat gynaecological disorders is selected from the group consisting of menopausal drugs like calcium carbonate and hormones; the active ingredient used to treat neoplastic disorders is selected from the group consisting of anti-neoplasics like letrozole; the active ingredient used to treat dependence is selected from the group consisting of anti-opioiate dependence drugs like buprenorphine and naloxone, anti-alcohol dependence drugs like acamprosate and anti-smoking dependence drugs like bupropion.

The man ordinary skilled in the art would find obvious that the herein above list is indicative only, and that many other active agents will fall within the scope of the present invention.

In yet another embodiment, the solid dosage form has a mass of 50 to 150 mg, with the active ingredient being present in the dosage form in an amount not exceeding 15 mg. The at least one first disintegration agent is present in the dosage form in an amount not exceeding 10% with respect to the total weight of the dosage form, while the second disintegration agent is present in the dosage form in an amount not exceeding 10% with respect to the total weight of said dosage form. In this embodiment, the first and the second disintegration agent are present in total amounts that provide a weight ratio of about 1:1 to about 1:2, wherein the dosage form provides at least one of the in vitro or in vivo disintegration time that is less than 10 seconds, and has a friability of 0.8% or less according to the U.S. Pharmacopoeia test when manufactured on rotary tableting machine operated at industrial speeds up to 60 rpm, and being obtained by direct compression.

In yet another embodiment, the solid dosage form may also have a mass of 150 to 300 mg, with the active ingredient being present in the dosage form in an amount not exceeding 50 mg. Here, the at least one first disintegration agent is present in the dosage form in an amount not exceeding 15% with respect to the total weight of the dosage form, and the second disintegration agent is present in the dosage form in an amount not exceeding 15% with respect to the total weight of the dosage form. The first and the second disintegration agent are present in total amounts that provide a weight ratio of about 1:1 to about 1:3, wherein the dosage form provides at least one of the in vitro or in vivo disintegration time that is less than 15 seconds, and has a friability of 0.8% or less according to the U.S. Pharmacopoeia test when manufactured on rotary tableting machine operated at industrial speeds up to 60 rpm, and being obtained by direct compression.

In yet another embodiment, the solid dosage form has a mass of 300 to 500 mg, with the active ingredient being present in the dosage form in an amount not exceeding 200 mg. In this case, the at least one first disintegration agent is present in the dosage form in an amount not exceeding 15% with respect to the total weight of the dosage form, and the second disintegration agent(s) is present in the dosage form in an amount not exceeding 15% with respect to the total weight of said dosage form. The first and the second disintegration agent are present in total amounts that provide a weight ratio of about 1:1 to about 1:3, wherein the dosage form provides at least one of the in vitro or in vivo disintegration time that is less than 20 seconds, and has a friability of 1% or less according to the U.S. Pharmacopeia test when manufactured on rotary tableting machine operated at industrial speeds up to 60 rpm, and being obtained by direct compression.

In yet another embodiment, the solid dosage form has a mass of 500 to about 1000 mg, with the active ingredient being present in the dosage form in an amount not exceeding 500 mg. The at least one first disintegration agent is present in the dosage form in an amount not exceeding 15% with respect to the total weight of the dosage form, and the second disintegration agent is present in the dosage form in an amount not exceeding 15% with respect to the total weight of the dosage form. The first and the second disintegration agent are present in total amounts that provide a weight ratio of about 1:1 to about 1:3, wherein the dosage form provides at least one of the in vitro or in vivo disintegration time that is less than 30 seconds, and has a friability of 1% or less according to the U.S. Pharmacopoeia test when manufactured on rotary tableting machine operated at industrial speeds up to 60 rpm, and being obtained by direct compression.

In another aspect of the present invention, there is provided a process for preparing the solid dosage formulation by directly compressing the components to form the formulation. Specifically, the method of manufacturing and packaging a formulation generally includes the steps of:

(i) blending the active ingredient(s) and the excipients, including at least the water-soluble disintegrant and the water-insoluble non swelling disintegrant; and

(ii) turning the blend into hard orally disintegrating tablets by direct compression; and

(iii) packaging the orally disintegrating tablets in blister foils or in bottles for instance.

In a preferred embodiment, formulations according to the present invention may be handled without any particular caution and may be packaged as oral tablets not intended for rapid oral dissolution/disintegration.

The present invention also relates to a method of rapidly administering an active ingredient by orally administering the solid dosage formulation to a patient in need thereof.

### BRIEF DESCRIPTION OF THE DRAWINGS

The present invention will be further described in the following description with references to the drawings in which:

FIG. 1 is a graphic illustration of the effect of compression force on the disintegration time of disintegrating formulations including various diluents;

FIG. 2 is a graphic illustration of the dissolution profiles of a formulation prepared according to the present invention over a storage period of three months; and

FIGS. 3-4 are graphic illustrations of the effect of press rotation speed on friability, hardness, and in vitro disintegration time of formulations prepared according to the present invention.

### DETAILED DESCRIPTION OF PREFERRED EMBODIMENTS

The present invention now provides an orally disintegrating dosage form having improved cohesion, i.e., having a friability value that is lower than a control formulation. A dosage form of the present invention fulfills the requirements of the United States Pharmacopoeia for tablet friability, i.e. “a maximum weight loss of not more than 1% of the weight of the tablets” (see USP 29-NF 24, paragraph <1216>“TABLET FRIABILITY”, page 3046-3047 for “conventional” tablets, i.e. those that are not intended to dissolve and/or to disintegrate within the buccal cavity within a reasonably time duration, e.g. less than 60 seconds for instance). In general, a maximum weight loss of not more than 0.8% to 1.0% is acceptable for most tablets. This is advantageous as the USP further explains that “effervescent tablets and chewable tablets may have different specifications as far as friability is concerned, and these tablets normally require special packaging.”

The inventors have demonstrated that combinations of polymers of acrylic type, namely the methacrylic acid copolymers of type C according to the USP/NF, and crospovidone or cross-linked polymer of povidone derivatives thereof, in certain ratios ranging from about 1:1 to about 1:3, are, unexpectedly, capable of very significantly improving the cohesion of the tablet, while maintaining a very good speed of disintegration. The use of the combination of the disintegrants according to the invention has the particular advantage of obtaining rapid disintegration tablets, and more particularly immediate-type disintegration tablets, which display very good friability allowing for no special caution to be taken when handling, packaging or dispensing the tablets.

The very low friability resulting from the very good cohesion greatly simplifies packaging of the tablets produced according to the invention since the tablets produced by means of the use or the process according to the invention are compatible with packaging intended for non-orally disintegrating dosage forms.

The low hygroscopicity of the formulations according to the present invention is also responsible for enhanced storage stability characteristics of said tablets over time.

The tablet disintegration effect observed according to the invention does not correspond to a simple erosion of mechanical type, but rather to an effect of the dissolution type after appropriate hydration of the tablet.

By “tablet disintegration agent” herein is meant an agent allowing an improvement in the disintegration speed observed for this tablet in the absence of this agent. This improvement in tablet disintegration speed can naturally be optimized by choosing the other tablet characteristics (such as type and quantity of the tablet's components, mass, format, hardness of the tablet) such that they do not oppose or even that they enhance the disintegration phenomenon.

By “rapid tablet disintegration agent” is thus understood herein as an agent offering a significant improvement in the tablet's disintegration speed, as indicated above. The term “significant” can be appreciated using any statistical tool known to a person skilled in the art. Appropriate conditions for observing this significant improvement include those which consist in placing said tablet in medium conditions and in particular in composition, pH and temperature conditions suited to the disintegration of the tablet in question. Water and simulated saliva fluids at a temperature of about 37° C. +/−1° C. are preferred conditions.

By “immediate type tablet disintegration agent” is understood herein as an agent allowing the disintegration of said tablet over a period lasting approximately 30 seconds or less, preferably approximately 20 seconds or less, even more preferably approximately 10 seconds or less, when the tablet is tested under conditions appropriate for its disintegration, and when the other components of the tablet and its structure (mass, format, hardness) are chosen in such a way that they do not oppose, or they even enhance, the disintegration phenomenon. Appropriate conditions for testing the disintegration of a tablet include conditions which mimic those under which said tablet is intended to break up. For example, in the case of a tablet intended to break up under the physiological conditions of a buccal cavity, such appropriate conditions include the fact of testing said tablet on an apparatus of Erweka ZT3™ type in a saliva medium at 37+/−1° C. and pH 6.0.

The term “agent” used herein also covers a co-agent situation. Thus the use according to the invention advantageously includes the use of a methacrylic acid copolymer of type C according to the USP/NF as an immediate type disintegration agent according to the invention, combined with the use of one or more known disintegration agents such as crospovidone (for example, those marketed under the trade mark KOLLIDON™ CL, KOLLIDON™ CL-F, KOLLIDON™ CL-SF, or KOLLIDON™ CL-M by BASF Aktiengesellschaft, Ludwigshafen, Germany, or those marketed under the trade mark POLYPLASDONE™ XL or POLYPLASDONE™ XL 10 by ISP, Wayne, N.J., USA).

The methacrylic acid copolymer(s) of type C used as disintegration agent(s) or co-agent(s) according to the invention can in particular be used for the production of any tablet requiring an improvement in the disintegration speed, and in particular a high disintegration speed. This is in particular the case for tablets adapted or intended for a pharmaceutical, veterinary or hygiene use. There can in particular be mentioned pharmaceutical, veterinary or hygiene tablets intended for administration by oral route for disintegration in the buccal cavity, and those intended for administration by oral route for deferred disintegration.

The buccal disintegration tablets can however have the drawback of an unpleasant taste and/or smell. In order to reduce or even eliminate the unpleasant smell and/or taste of the active ingredient(s), active ingredient(s) can be coated. Obviously, the methacrylic acid copolymer(s) of type C used as disintegration agent(s) or co-agent(s) according to the invention can not be used as a coating agent as well, since it will dissolve readily when placed in the buccal cavity of the patient, hence releasing the bad-tasting and/or bad-smelling agent.

The proportions in which said combinations of methacrylic acid copolymer(s) of type C and crospovidone derivative(s) must be used according to the invention can easily be tested by trial and error using techniques known to a person skilled in the art, according to the complete formulation of the tablet chosen, and according to the effect sought. For example, these proportions are generally comprised between approximately 5 and 15% of the total mass of the tablet, in ratios ranging from 1:1 to 1:3, depending on the tablet mass, the active ingredient loading, among many other parameters known by one of ordinary skill in the art.

The methacrylic acid copolymer(s) of type C implemented, or used as disintegration agent(s) or co-agent(s) according to the invention do not cause any restriction in the possible nature of the other elements of the dosage form, except for the choice of a coating agent as explained previously. It or they can thus be combined with any substance or excipient appropriate to the type of application for which the tablet is intended.

In a quite general manner, formulations according to the invention further comprise the use of an active ingredient or a placebo, the use of a diluent, and the use of a lubricant. More particularly, formulations according to the invention can also further comprise the use of excipients or substances playing the following roles:
water-soluble diluents. Sugar alcohols such as sorbitol, mannitol, xylitol, maltitol, lactitol, etc. . . . are preferred water-soluble diluents. Among sugar alcohols, mannitol is the most preferred. Preferably, mannitol is the substantially sole diluent used. However, mannitol may be the primary diluent if the formulations of the present invention also contain another water-soluble diluent(s) in a small amount. Importantly, insoluble diluents such as, for instance, calcium carbonates, calcium phosphates, calcium sulfates, kaolin, magnesium carbonates, magnesium oxide, and talc lactose or simple sugars such as fructose are not used as diluent in formulations according to the invention.
lubricants such as magnesium stearate,
flowability regulator agents such as colloidal silica,
solubilization agents,
flavors and flavor enhancers
sweeteners,
plasticizers,
preservatives and antioxidants,
film forming and coating agents,
agents involved in the composition of the polishing and shining solution,
agents providing thermal protection of the active ingredient such as saccharose derivatives,
excipients or substances providing bioadhesion such as acrylic acid derivatives, the copolymer of methylvinylether and maleic anhydride, guar gum, xanthane gum, carouba, carraghenates, pectin, a biological or synthetic protein alone or in combination with other proteins of biological or synthetic origin, cyclodextrins, hydroxypropylbetacyclodextrins, betacyclodextrins and their derivatives.

The skilled artisan would find obvious that the herein above list is indicative only, and that many other ingredients will fall within the scope of the present invention.

The use according to the invention allows any desired active ingredient to be combined with the tablet. There can in particular be mentioned antihistamines, anticholinergics, mineral elements, allergens, surface, local or general anesthetics, antipyretics, non-opiate analgesics, opiate analgesics, anticholinergic and non-anticholinergic antispasmodics, non-steroid anti-inflammatories such as tiaprofenic acid, indomethacin, diclofenac, ibuprofen, ketoprofen, naproxen, piroxicam, steroid anti-inflammatories such as betamethasone, prednisolone, cytotoxics, antihormonal agents, antianaemics, antiemetics, antiasthenics, antihypertensives including beta-blockers such as propranolol, atenolol, metoprolol, conversion enzyme inhibitors such as captopril, enalapril, angiotensin II antagonists, calcium inhibitors such as nifedipine and diltiazem, central action antihypertensives, vasodilators, hypolipemiants, oral antidiabetics, anticoagulants, platelet antiaggregants, calcium inhibitors, nitrated derivatives used in the treatment of coronary insufficiency, non-nitrated antianginals, diuretics, digitalin derivatives and related derivatives, antiarhythmics, antihypotensives and circulatory analeptics, vasodilators, anti-ischemics, vasculoprotectors and venotonics, hormones, antiherpetics, antiphotosensitizers, antiulceratives such as ranitidine, cimetidine, antacids, laxatives, antidiarrheals, antifungals, cholelitholytics, interferons, enzymes, antispasmodics, antibacterials, antiseptics, antiherpetics, uterorelaxants, oxytocics, oestrogens, progestatives, oestroprogestatives, the active ingredients indicated in lactation such as bromocriptine, the active ingredients indicated in the treatment of sterility, antigonadotropics, anticoagulants, thrombolytics, antifibrinolytics, vitamins, haemostatics, cyclosporines, alkylating agents, antibiotics, antivirals, antiparasitics, vaccines, diagnostic products, the active ingredients indicated in the treatment of obesity, orexigenics, the active ingredients indicated in the treatment of the correction of metabolic abnormalities, the active ingredients indicated in oral and enteral nutrition, anticonvulsives, antiparkinsonians, antimyasthenics, the active ingredients indicated in the treatment of Alzheimer's disease, antimigraine agents, neuroleptics, anxiolytics, hypnotics, sedatives, antidepressants, normothymrics, psychostimulants, the active ingredients indicated in the treatment of states of alcohol addiction, tobacco disintoxication, opiate disintoxication, antiglaucoma agents, mydriatics, bronchodilators, antiasthmatics, antitussives, bronchial fluidifiers, (topical) revulsives, the active ingredients indicated in the treatment of osteopathies, the active ingredients indicated in the treatment of acute attacks of gout, the active ingredients indicated in the treatment of hypouricemia, the active ingredients indicated in the treatment of algodystrophiae, myorelaxants, the active ingredients indicated in the treatment of arthrosis, correctors of hyposialoses, the active ingredients indicated in the treatment of urinary lithiasis, the active ingredients indicated in the treatment of renal insufficiency, the active ingredients indicated in the treatment of enuresis, the active ingredients indicated in the treatment of retrograde ejaculation, the active ingredients indicated in the treatment of impotence.

The combination of methacrylic acid copolymer(s) of type C and crospovidone or derivatives thereof according to the invention can be incorporated by mixture into the tablet mass, or can only be part of certain sub-structures of the tablet, for example be incorporated with micro- or nano-particles (or micro- or nano-capsules) included in a tablet, and/or be incorporated into a layer of a multi-layer tablet, in particular in a layer intended for rapid disintegration. This tablet, intended for rapid disintegration, whether mono-layered, particular, multi-layered or a combination of these arrangements, is advantageously presented in the form of a bioadhesive tablet, and/or a tablet for the rapid but deferred release of the active ingredient(s) (for example, rapid disintegration at the level of the intestines after administration by oral route), or a tablet for the rapid and immediate release of the active ingredient(s) (for example, rapid disintegration in the mouth).

The at least one methacrylic acid copolymer of type C and the crospovidone or derivative thereof are present in a weight ratio of about 1:1 to about 1:3.

Advantageously, the use of tablet formulations according to the invention comprises a use of said tablet mass (or, where appropriate, of said tablet sub-structure) in an essentially pulverulent form before being turned into a dosage form using any technique known to a person skilled in the art, such as wet granulation, dry granulation and compaction, extrusion, as well as, advantageously, direct compression. Restriction to one or more types of techniques can be observed according to the nature and/or proportion of the other components used in the production of said tablet, and/or according to the structure to be given to this tablet. Restrictions will be appreciated by the man ordinary skilled in the art. Preferentially, the use according to the invention comprises turning said tablet, or, where appropriate, said tablet sub-structure into dosage form, by simple direct compression. Remarkably, the use according to the invention allows tablets to be obtained which have very good pharmacotechnical characteristics and which in particular, after being turned into a dosage form, display a friability of 1% or less according to USP29/NF24.

The use according to the invention is suited to the production of tablets of any mass and any format, without limitation.

Particularly remarkably, the use according to the invention allows tablets to be obtained which, while having very good pharmacotechnical characteristics, are capable of breaking up in a time period of less than approximately 30 seconds, preferably in a time period less than or equal to approximately 20 seconds, more preferably less than or equal to approximately 15 seconds, and even more preferably less than or equal to approximately 10 seconds when they are placed in appropriate conditions for their disintegration. The determination of appropriate conditions is known to a person skilled in the art, and examples of this are given herein.

Examples of methacrylic acid copolymers of type C according to the USP/NF which can be used according to the invention include those marketed by the company BASF (Ludwigshafen, Germany) under the name KOLLICOAT™ MAE 100 P (pulverulent form), or KOLLICOAT™ MAE 30 DP (aqueous dispersion), or by the company Rohm GmbH (Darmstadt, Germany), under the name EUDRAGI™ L100-55 (pulverulent form), or EUDRAGIT™ L30D-55 (aqueous dispersion). EUDRAGIT™ L100-55 corresponds to the following formula:

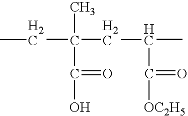

A subject of the present invention is also a production process for the tablets, in particular rapid disintegration tablets, the process involving the use of combinations of at least one methacrylic acid copolymer of type C according to the USP/NF as a disintegration agent or co-agent and crospovidone or derivatives thereof. More particularly, the at least one methacrylic acid copolymer of type C used as a disintegrant according to the invention has no function of coating the active ingredient, such as enteric coating (for sustained-release for instance), insulation coating (such as for protecting the active from tropical conditions for instance), or taste-masking and/or smell masking coating. Preferentially and advantageously, the process according to the invention further comprises turning the tablets into dosage form by simple direct compression.

### EXAMPLES

The following examples are given for illustration purposes, but do not in any way limit the invention. In these examples, different pharmacotechnical parameters are measured using standard techniques. Among these parameters there can in particular be mentioned hardness, friability as well as stability of the tablet obtained according to the use and/or the process of the invention. Among the means available to a person skilled in the art for measuring such parameters, there can be mentioned:
a Erweka TBH30 type apparatus for measuring hardness,
a USP friability apparatus for measuring the friability and a suitable friability measurement protocol, as described in USP 29-NF 24, paragraph <1216>“TABLET FRIABILITY”, page 3046-3047,
a tablet disintegration apparatus of Erweka ZT3 type using a suitable measurement protocol which includes placing 1 tablet in each of 6 glass tubes in a basket. The basket is then suspended and maintained above the beaker filled with water or saliva medium. The disintegration time is measured from the time the basket is lowered into the beaker until total disintegration of the tablets. Composition of the saliva medium used (pH=6): KCl: 1.20 g/l; MgCl₂, 6H₂O: 0.05 g/l; CaCl₂, 6H₂O: 0.15 g/l; KSCN: 0.10 g/l.

These and other embodiments of the present invention will readily occur to those of ordinary skill in the art in view of the examples herein after.

The following examples are put forth so as to provide those of ordinary skill in the art with a complete disclosure and description of how to make and use the formulations, methods, and devices of the present invention, and are not intended to limit the scope of what the inventors regard as the invention. Efforts have been made to ensure accuracy with respect to numbers used (e.g., weights, temperature, volumes, etc.) but some experimental errors and deviations should be accounted for. Unless indicated otherwise, parts are parts by weight, molecular weight is weight average molecular weight, temperature is in degrees Centigrade, and pressure is at or near atmospheric.

The compositions produced according to the present invention meet the strict specifications for content and purity required of pharmaceutical products.

### Example 1

Examples 2, 3, 4 and 5 of U.S. Pat. No. 6,696,085 contain a small amount of sorbitol (about 3.00% by weight of the total tablet) in order to “improve the compressibility of the mixture ofpowders”. Addition of sorbitol consequently results in the possibility to “work with lower hardnesses while obtaining a tablet which is not very friable”.

Sorbitol has been further added in one “EUDRAGIT” formula falling within the scope of the invention of U.S. Pat. No. 6,696,085 until totally replacing dextrose. Tablets were produced by direct compression on the same alternative tableting machine (KORSCH EK0) than in the U.S. Pat. No. 6,696,085, following the “EUDRAGIT” formula indicated in Table 1 herein.

Tablets were produced targeting USP friability not higher than 1%.

The tablets are of a flat, bevel-edged shape. Diameter is 7 mm. Mass is about 100 mg.

TABLE 1

| Excipients | Content (% w/w) |
|---|---|
| EUDRAGIT L 100-55 ® (Röhm) | 10.0% |
| KOLLIDON CL ® (BASF) | 10.0% |
| Sorbitol Neosorb P 60 W (Roquette) | 69.5% |
| Lemon 501163 TP0551 | 7.00% |
| Aspartame fine powder | 1.50% |
| Citric acid anhydrous | 1.00% |
| Magnesium stearate | 1.00% |

The pharmacotechnical parameters, and in particular the average hardness, the average friability and the average in vitro disintegration time for these tablets were then measured. This “sorbitol” formulation was easily compactable and allowed decreasing the compression force giving tablets with a low hardness (about 11 Newton, from 10N to 13N) while passing the USP friability test (friability: 0.57%).

However, in vitro disintegration time (about 37 seconds) obtained was not acceptable for a dosage form intended for oral disintegration. Thus it appears that rapid in vitro disintegration time of the formulation recited in Example 2 of the U.S. Pat. No. 6,696,085 patent was obtained to the detriment of the friability.

### Examples 2 and 3

Because of the poor disintegration properties of the “sorbitol” formulation of Example 1, sorbitol was then replaced by another sugar alcohol not promoting tooth decay. Mannitol was selected. Flavors, sweeteners and flavor enhancers were further withdrawn for better discrimination of the results. Tablets were produced by direct compression on the same alternative tableting machine (KORSCH EK0) as in the U.S. Pat. No. 6,696,085, following the “EUDRAGIT” formula indicated in Table 2 herein.

Tablets were produced targeting USP friability not higher than 1%.

The tablets are of a flat, bevel-edged shape. Diameter is 7 mm. Mass is about 100 mg.

TABLE 2

Content (% w/w)

| Components | Example 2 | Example 3 |
|---|---|---|
| EUDRAGIT L 100-55 ® (Rhöm) | 10.0 | 10.0 |
| KOLLIDON CL ® (BASF) | 10.0 | 10.0 |
| Sorbitol Neosorb P 60 W (Roquette) | 79.0 | — |
| Mannitol PEARLITOL 200SD | — | 79.0 |
| (Roquette) | | |
| Magnesium stearate | 1.00 | 1.00 |

The pharmacotechnical parameters, and in particular the average hardness, the average friability and the average in vitro disintegration time for these tablets were then measured. The hardness and disintegration time results for these tablets are set out in Table 3 herein.

TABLE 3

| | Hardness | In vitro disintegration time | USP friability |
|---|---|---|---|
| Formula | (N) | (sec) | (%) |
| Example 2 | 19.7 | 114 (1 minute 54 seconds) | 0.47 |
| Example 3 | 22.4 | 10 | 0.34 |

Replacing total sorbitol by mannitol allows obtaining fast orally disintegrating dosage forms passing the USP friability test.

### Examples 4 and 5

Because of the very positive effect of the substitution of sorbitol by mannitol on both in vitro disintegration time and friability in the previous examples, it could be hypothesized that all the benefit is attributable to mannitol and that, consequently, the presence of disintegrant of the present invention in the formulation may be useless. Therefore formulations containing only mannitol (Example 4), mannitol and type-C polymethycrylate (Example 5) were manufactured. Tablets were produced by direct compression on the same alternative tableting machine (KORSCH EK0) as in the U.S. Pat. No. 6,696,085, following the formula indicated in Table 4 herein.

Tablets were produced targeting USP friability not higher than 1%.

The tablets are of a flat, bevel-edged shape. Diameter is 7 mm. Mass is about 100 mg.

TABLE 4

Content (% w/w)

| Components | Example 4 | Example 5 |
|---|---|---|
| EUDRAGIT L 100-55 ® (Röhm) | — | 10.0 |
| Mannitol PEARLITOL 200SD | 99 | 89.0 |
| (Roquette) | | |
| Magnesium stearate | 1.00 | 1.00 |

The pharmacotechnical parameters, and in particular the average hardness, the average friability and the average in vitro disintegration time for these tablets were then measured. The hardness and disintegration time results for these tablets are set out in Table 5 herein.

TABLE 5

| | Hardness | In vitro disintegration time | USP friability |
|---|---|---|---|
| Formula | (N) | (sec) | (%) |
| Example 4 | 21.2 | 204 (3 minute 24 seconds) | 0.69 |
| Example 5 | 19.7 | 30 | 0.60 |

Addition of type-C polymethacrylate to a formulation containing only mannitol allows decreasing significantly the time for in vitro disintegration while obtaining oral dosage forms passing the USP friability test.

Further addition of crospovidone (so that type-C polymethacrylate and crospovidone are present in a 1:1 ratio, example 3) allows for an even faster in vitro disintegration (10 seconds). Outstandingly, friability of the example 3 (0.34%) is also better than the friability of the control, i.e. not containing the type-C polymethacrylate (0.41%).

### Examples 6, 7 and 8

U.S. Pat. No. 6,696,085 claims ratios of 1:10 to about 1:1 to about 50:1, ratios between 50:1 to 1:1 being preferred, and the ratio 2:1 being the most preferred. Various ratios of type-C polymethacrylate to crospovidone have been investigated and were compared to Example 3.

Tablets were produced by direct compression on the same alternative tableting machine (KORSCH EK0) as in the U.S. Pat. No. 6,696,085, following the formula indicated in Table 6 herein.

Tablets were produced targeting USP friability not higher than 1%.

The tablets are of a flat, bevel-edged shape. Diameter is 7 mm. Mass is about 100 mg.

TABLE 6

| | Content (% w/w) | | | |
|---|---|---|---|---|
| Components | Example 3 | Example 6 | Example 7 | Example 8 |
| EUDRAGIT L 100- | 10.0 | 5.00 | 10.0 | 5.00 |
| 55 ® (Röhm) | | | | |
| KOLLIDON CL ® | 10.0 | 5.00 | 5.00 | 10.0 |
| (BASF) | | | | |
| Mannitol PEARLITOL | 79.0 | 89.0 | 84.0 | 84.0 |
| 200SD (Roquette) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. The hardness and disintegration time results for these tablets are set out in Table 7 herein.

TABLE 7

| | Hardness | In vitro disintegration time | USP friability |
|---|---|---|---|
| Formula | (N) | (sec) | (%) |
| Example 3 | 30.8 | 9.3 | 0.47 |
| Example 6 | 31.1 | 9.8 | 0.45 |
| Example 7 | 25.8 | 11.3 | 0.40 |
| Example 8 | 31.1 | 9.6 | 0.30 |

Example 7 (2:1 ratio) presents a disintegration time higher than 10 seconds, despite exhibiting the lowest hardness (about 20% lower than the three other formulations). This means that compressed at a force so that hardness would be similar to the one of the three other examples, friability may be improved but to the detriment of in vitro disintegration time, which would be even longer.

Outstandingly, type-C polymethacrylate to crospovidone ratios of 1:1 to 1:2 allows obtaining in vitro disintegration time less than 10 seconds.

### Examples 9, 10, 11, 12 and 13

Various direct-compression sugar alcohols, beside mannitol and sorbitol, have been investigated.

Tablets were produced by direct compression on a single-punch alternative tableting machine (KILIAN) instrumented with a piezoelectric load washer for force compression monitoring.

The tablets are of a flat-shaped. Diameter is 8.1 mm. Mass is about 110 mg.

TABLE 8

| | Content (mg) | | | | |
|---|---|---|---|---|---|
| | | Example | Example | Example | Example |
| Components | Example 9 | 10 | 11 | 12 | 13 |
| EUDRAGIT L 100-55 ® | 10.0 | 10.0 | 10.0 | 10.0 | 10.0 |
| (Röhm) | | | | | |
| KOLLIDON CL ® | 10.0 | 10.0 | 10.0 | 10.0 | 10.0 |
| (BASF) | | | | | |
| Mannitol PEARLITOL 200SD | 80.0 | — | — | — | — |
| (Roquette) | | | | | |
| Isomalt DC100 (Tillmanns) | — | 80.0 | — | — | — |
| Xylitol Xylitab ® (Roquette) | — | — | 80.0 | — | — |
| Maltitol Maltisorb P200 ® | — | — | — | 80.0 | — |
| (Roquette) | | | | | |
| Sorbitol Neosorb DC ® | | | | | 80.0 |
| (Roquette) | | | | | |
| Citric acid | 0.50 | 0.50 | 0.50 | 0.50 | 0.50 |
| Mint flavor (Givaudan) | 7.00 | 7.00 | 7.00 | 7.00 | 7.00 |
| Sweetener SUCRAM PH821 ® | 0.80 | 0.80 | 0.80 | 0.80 | 0.80 |
| (Pancosma) | | | | | |
| Silica Syloid 244 ® (Grace) | 1.50 | 1.50 | 1.50 | 1.50 | 1.50 |
| Magnesium stearate | 0.60 | 0.60 | 0.60 | 0.60 | 0.60 |

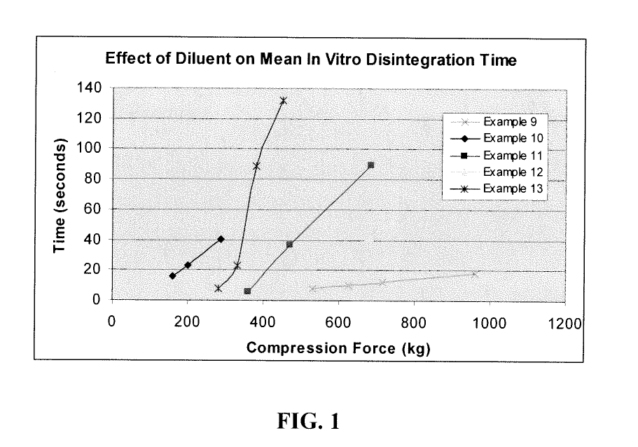

The pharmacotechnical parameters, and in particular the average hardness, the average friability and the average in vitro disintegration time for these tablets were then measured. The hardness and disintegration time results for these tablets are set out in FIG. 1.

The ability of an orally disintegrating dosage form to withstand changes in compression force during production and to maintain acceptable in vitro disintegration time can be expressed by comparing the slopes of linear regression relationships between Compression Force (X) and In Vitro Disintegration Time (Y), as set out in Table 9 here herein.

TABLE 9

| | Slope | R-squared value |
|---|---|---|
| Example 9 | Y = 0.0236X − 4.5639 | 0.9985 |
| Example 10 | Y = 0.1896X − 14.559 | 0.9997 |
| Example 11 | Y = 0.2534X − 84.042 | 0.9994 |
| Example 12 | Y = 0.1775X − 78.776 | 0.9999 |
| Example 13 | Y = 0.7804X − 217.94 | 0.9563 |

Mannitol presents the lowest value of slope, meaning that a change in compression force, likely to happen during industrial production runs, would not result in a significant change in in-vitro disintegration time.

This is not the case at all for the other excipients herein tested, thereby supposing that compression force should be continuously monitored and very finely tuned so that in-vitro disintegration time is not dramatically impaired.

### Example 14

An active ingredient (meloxicam, a non-steroidal anti inflammatory) is added to a formulation of the present invention since this could dramatically modify the observations made so far on “placebo” tablets.

Tablets were produced by direct compression on the same alternative tableting machine (KORSCH EK0) as in the U.S. Pat. No. 6,696,085, following the “EUDRAGIT” formula indicated in Table 10 herein.

Tablets were produced targeting USP friability not higher than 1%.

The tablets are of a flat, bevel-edged shape. Diameter is 7 mm. Mass is about 115 mg.

TABLE 10

| Components | Content (% w/w) |
|---|---|
| Meloxicam | 7.50 mg |
| Polymethacrylate - EUDRAGIT L 100-55 | 6.00 mg |
| Crospovidone - KOLLIDON CL | 12.0 mg |
| Mannitol - PEARLITOL 200SD | 83.9 mg |
| Lemon flavor | 2.50 mg |
| Aspartame | 1.00 mg |
| Citric acid | 1.00 mg |
| Stearate Mg | 1.10 mg |

The pharmacotechnical parameters, and in particular the average hardness, the average friability and the average in vitro disintegration time for these tablets were then measured. The hardness and disintegration time results for these tablets are set out in Table 11 herein.

TABLE 11

| | Hardness | In vitro disintegration time | USP friability |
|---|---|---|---|
| Formula | (N) | (sec) | (%) |
| Example 14 | 24.1 | 7.5 | 0.26 |

### Example 15

Tablets of Example 14 were packed in plastic bottles and stored in a climatic chamber (40° C./25% R.H.) for a period of three months.

The pharmacotechnical parameters, and in particular the average friability and the average in vitro disintegration time for these tablets were then measured each month. Results are set out in Table 12 herein.

TABLE 12

| | In vitro disintegration time | USP friability |
|---|---|---|
| Time point | (sec) | (%) |
| T0 | 7.5 | 0.26 |
| T1 month | 8.3 | 0.15 |
| T2 month | 7.7 | 0.13 |
| T3 month | 8.7 | 0.33 |

### Example 16

Tablets of Example 14 were packed in plastic bottles and stored in a climatic chamber (40° C./25% R.H.) for a period of three months.

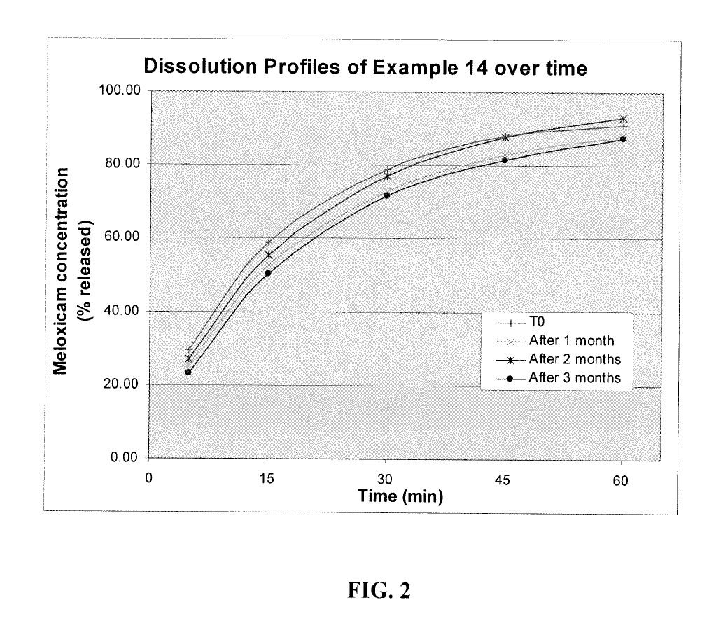

The dissolution profiles were then generated each month. Results are set out in FIG. 2. Release of the active ingredient was maintained constant over time, as demonstrated by the dissolution profile patterns here above.

### Example 17

A formulation of the present invention was manufactured at industrial scale (batch of about 40,000 tablets); in order to validate laboratory data generated so far (batch size of about 1,000 tablets).

Tablets were produced by direct compression on an instrumented rotary 10-station tableting machine (PICCOLA), following the “EUDRAGIT” formula indicated in Table 13 herein.

Tablets were produced targeting USP friability not higher than 1%.

The tablets are of a flat, bevel-edged shape. Diameter is 7 mm. Mass is about 115 mg.

TABLE 13

| | |
|---|---|
| Components | Content (mg) |

| | | |
|---|---|---|
| Meloxicam | 15.0 | mg |
| Polymethacrylate - EUDRAGIT L 100-55 | 5.00 | mg |
| Crospovidone - KOLLIDON CL | 10.0 | mg |
| Mannitol - PEARLITOL 200SD | 74.9 | mg |
| Flavor | qs 115.0 | mg |
| Flavor enhancer | | |
| Sweetener | | |
| Lubricant | | |

The pharmacotechnical parameters, and in particular the average hardness, the average friability and the average in vitro disintegration time for these tablets were then measured varying compression forces. Results are set out in Table 14 herein.

TABLE 14

| | Press | Average | Average In vitro | Average |
|---|---|---|---|---|
| Compression | Speed | Hardness | disintegration | USP |
| Force (KN) | (rpm) | (N) | time (sec) | friability (%) |
| Between 5 and 6 | 20 | 9.8 | Between 9 and 10 | 0.29 |
| Between 8 and 9 | 25 | 20.6 | Between 8 and 9 | 0.24 |

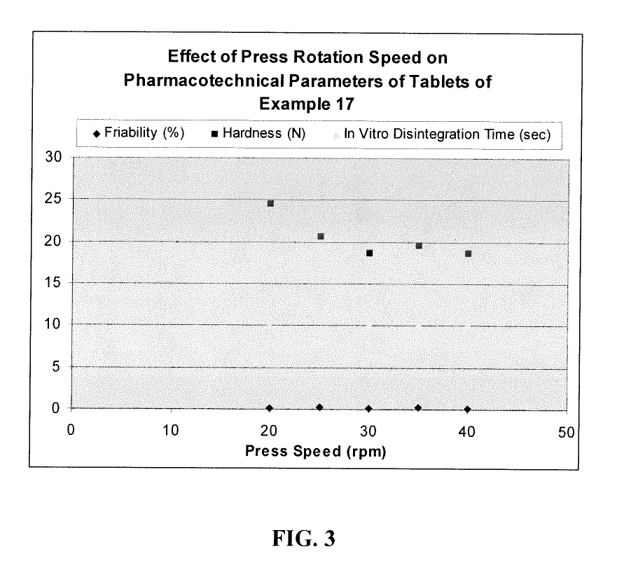

Rotary press was operated at various speeds. Effect of press rotation speed on pharmacotechnical parameters is shown in FIG. 3.

Hardness is maintained almost constant (average: 20.4N+/−N) over the 20 rpm-40 rpm rotation speed range, as well as friability (average: 0.184%+/−0.04%) and in vitro disintegration times (average: 9′6 seconds +/−0′9).

The same formulation as disclosed in Table 13 hereinabove is now scaled up to 120,000-tablet batch size, using an instrumented rotary 36-station tableting machine (KIKUSUI), operated at 30 rpm. The tablets are of a flat, bevel-edged shape. Diameter is 7 mm. Mass is about 115 mg. The pharmacotechnical parameters, and in particular the average hardness, the average friability and the average in vitro disintegration time for these tablets were then measured every ten minutes as In Process controls. Results are set out in Table 15 herein.

TABLE 15

| | Press | Average | Average In vitro | |
|---|---|---|---|---|
| Run Time | Speed | Hardness (N) | disintegration | Average USP |
| (minutes) | (rpm) | (min–max) | time (sec) | friability (%) |
| 0 | 30 | 17.5 (n = 10) | 5.7 sec (n = 5) | 0.32 |
| | | (14–25) | (min: 5 sec; | |
| | | | max 7 sec) | |
| 10 min | 30 | 16.2 (n = 5) | Not determined | Not determined |
| | | (15–18) | | |
| 20 min | 30 | 24.2 (n = 5) | Not determined | Not determined |
| | | (22–26) | | |
| 30 min | 30 | 20.8 (n = 5) | Not determined | Not determined |
| | | (19–23) | | |
| 40 min | 30 | 22.0 (n = 5) | Not determined | Not determined |
| | | (21–23) | | |
| 50 min | 30 | 17.4 (n = 10) | 5.2 sec (n = 6) | 0.19 |
| | | (15–20) | (min: 2 sec; | |
| | | | max 8 sec) | |
| 60 min | 30 | 19.8 (n = 5) | Not determined | Not determined |
| | | (17–23) | | |
| 70 min | 30 | 16.4 (n = 5) | Not determined | Not determined |
| | | (14–19) | | |
| 80 min | 30 | 17.8 (n = 5) | 5.3 sec (n = 6) | 0.18 |
| | | (13–20) | (min: 2 sec; | |
| | | | max 8 sec) | |
| 90 min | 30 | 17.8 (n = 5) | Not determined | Not determined |
| | | (16–19) | | |
| 100 min | 30 | 18.8 (n = 5) | Not determined | Not determined |
| | | (16–20) | | |
| 110 min | 30 | 19 (n = 8) | 6.2 sec (n = 6) | 0.32 |
| | | (18–20) | (min: 3 sec; | |
| | | | max 9 sec) | |

These data further demonstrate the ability of a formulation of the present invention to be scaled up at industrial scale. Indeed, hardness is maintained almost constant (average: 18.7N+/−2.9N) at 30-rpm rotation speed, as well as friability (average: 0.25%+/−0.08%) and in vitro disintegration times (average: 5′6 seconds +/−1′8).

### Example 18

Another formulation of the present invention was manufactured at industrial scale (batch of about 40,000 tablets), in order to validate laboratory data generated so far (batch size of about 1,000 tablets).

Tablets were produced by direct compression on an instrumented rotary 10-station tableting machine (PICCOLA), following the “EUDRAGIT” formula indicated in Table 16 herein.

Tablets were produced targeting USP friability not higher than 1%.

The tablets are of a flat, bevel-edged shape. Diameter is 7 mm. Mass is about 115 mg.

TABLE 16

| Components | Content (mg) | |
|---|---|---|
| Meloxicam | 7.50 | mg |
| Polymethacrylate - EUDRAGIT L 100-55 | 6.00 | mg |
| Crospovidone - KOLLIDON CL | 12.0 | mg |
| Mannitol - PEARLITOL 200SD | 83.9 | mg |
| Flavor | qs 115.0 | mg |
| Flavor enhancer | | |
| Sweetener | | |
| Lubricant | | |

The pharmacotechnical parameters, and in particular the average hardness, the average friability and the average in vitro disintegration time for these tablets were then measured varying compression forces. Results are set out in Table 17 herein.

TABLE 17

| | Press | Average | Average In vitro | |
|---|---|---|---|---|
| Compression | Speed | Hardness | disintegration | Average USP |
| Force (KN) | (rpm) | (N) | time (sec) | friability (%) |
| About 5.5 | 30 | 22.8 | Between 5 and 8 | 0.18 |
| About 5.7 | 40 | 16.7 | Between 6 and 7 | 0.09 |
| About 5.7 | 50 | 19.1 | Between 5 and 7 | 0.09 |

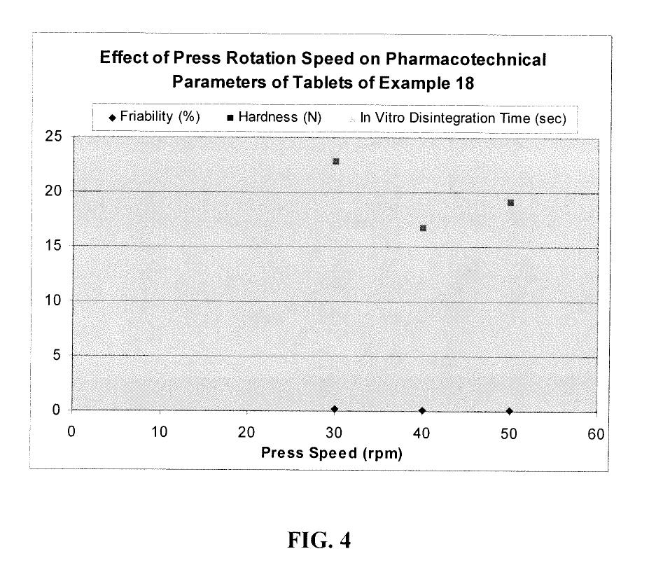

Rotary press was operated at various speeds. Effect of press rotation speed on pharmacotechnical parameters is shown in FIG. 4.

### Example 19

A formulation of the present invention containing Clonazepam, a benzodiazepine drug, was also investigated.

Tablets were produced by direct compression on the same alternative tableting machine (KORSCH EK0) as in the U.S. Pat. No. 6,696,085, following the “EUDRAGIT” formula indicated in Table 18 herein.

Tablets were produced targeting USP friability not higher than 1%.

The tablets are of a flat, bevel-edged shape. Diameter is 7 mm. Mass is about 100 mg.

TABLE 18

| Components | Content (% wt) |
|---|---|
| Clonazepam | 1.00 |
| Polymethacrylate - EUDRAGIT L 100-55 | 6.00 |
| Crospovidone - KOLLIDON CL | 12.0 |
| Mannitol - PEARLITOL 200SD | 77.8 |
| Flavor and flavor enhancer | qs 100.0 |
| Sweetener | |
| Lubricant | |

The pharmacotechnical parameters, and in particular the average hardness, the average friability and the average in vitro disintegration time for these tablets were then measured. Results are set out in Table 19 herein.

TABLE 19

| | | In vitro | |
|---|---|---|---|
| | Hardness | disintegration time | USP friability |
| Formula | (N) | (sec) | (%) |
| Example 19 | 23.7 | 7.8 | 0.33 |

### Examples 20, 21, 22 and 23

Examples recited so far relate to tablets with a mass of 150 mg or less. Formulations of the present invention with a tablet mass of 300 mg were also investigated.

Tablets were produced by direct compression on the same alternative tableting machine (KORSCH EK0) as in the U.S. Pat. No. 6,696,085, following the “EUDRAGIT” formula indicated in Table 20 herein.

Tablets were produced targeting USP friability not higher than 1%.

The tablets are of a flat, bevel-edged shape. Diameter is 11 mm. Mass is about 300 mg.

TABLE 20

| | Content (% wt) | | | |
|---|---|---|---|---|
| | Example | Example | Example | Example |
| Components | 20 | 21 | 22 | 23 |
| EUDRAGIT L 100-55 ® | — | 10.0 | — | 10.0 |
| (Rohm) | | | | |
| KOLLIDON CL ® | — | — | 10.0 | 10.0 |
| (BASF) | | | | |
| Mannitol PEARLITOL | 98.0 | 88.0 | 88.0 | 78.0 |
| 200SD (Roquette) | | | | |
| Mint flavor (Givaudan) | 0.50 | 0.50 | 0.50 | 0.50 |
| Sweetener SUCRAM | 0.50 | 0.50 | 0.50 | 0.50 |
| PH821 ® (Pancosma) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. Results are set out in Table 21 herein.

TABLE 21

| | | In vitro | USP |
|---|---|---|---|
| | Hardness | disintegration time | friability |
| Formula | (N) | (sec) | (%) |
| Example 20 | 22.4 | 49.7 | Failed |
| Example 21 | 21.9 | 35.5 | 0.75 |
| Example 22 | 21.7 | 16.5 | 0.60 |
| Example 23 | 20.5 | 11.4 | 0.79 |

As observed in Examples 4, 5 and 6 on tablets having a diameter of 7 mm and a mass of 100 mg, addition of type-C polymethacrylate (Example 21) to a formulation containing only mannitol (reference, Example 20) allows decreasing significantly the time for in-vitro disintegration while obtaining oral dosage forms passing the USP friability test.

Further addition of crospovidone (so that type-C polymethacrylate and crospovidone are present in a 1:1 ratio, Example 23) allows for an even faster in vitro disintegration (less than 15 seconds, at 11.4 seconds).

Noteworthy, crospovidone as the only disintegrant (Example 22) did not enable to obtain tablets disintegrating within less than 15 seconds in vitro. Outstandingly, friability of Example 23 (0.79%) is also better than the friability of the control, i.e. not containing the type-C Polymethacrylate, which failed the USP tests (several tablets broke during the test).

### Examples 24, 25, 26 and 27

Formulations of the present invention with a tablet mass of 300 mg varying the ratio of the two disintegrants/co-disintegrants were investigated.

Tablets were produced by direct compression on the same alternative tableting machine (KORSCH EK0) as in the U.S. Pat. No. 6,696,085, following the “EUDRAGIT” formula indicated in Table 22 herein.

Tablets were produced targeting USP friability not higher than 1%.

The tablets are of a flat, bevel-edged shape. Diameter is 11 mm. Mass is about 300 mg.

TABLE 22

| | Content (% wt) | | | |
|---|---|---|---|---|
| | Example | Example | Example | Example |
| Components | 24 | 25 | 26 | 27 |
| EUDRAGIT L 100-55 ® | 5.00 | 10.0 | 5.00 | 30.0 |
| (Rohm) | | | | |
| KOLLIDON CL ® | 10.0 | 5.00 | 15.0 | 15.0 |
| (BASF) | | | | |
| Mannitol PEARLITOL | 83.0 | 83.0 | 78.0 | 53.0 |
| 200SD (Roquette) | | | | |
| Mint flavor (Givaudan) | 0.50 | 0.50 | 0.50 | 0.50 |
| Sweetener SUCRAM | 0.50 | 0.50 | 0.50 | 0.50 |
| PH821 ® (Pancosma) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. Results are set out in Table 23 herein.

TABLE 23

| | | In vitro | USP |
|---|---|---|---|
| | Hardness | disintegration time | friability |
| Formula | (N) | (sec) | (%) |
| Example 24 | 21.5 | 10.7 | 0.60 |
| Example 25 | 22.4 | 15.7 | 0.84 |
| Example 26 | 20.4 | 9.7 | 0.79 |
| Example 27 | 19.4 | 9.0 | 1.65 |

Example 24 (EUDRAGIT: KOLLIDON ratio at 1:2) presents a friability of 0.60%, while friability of Example 25 (EUDRAGIT: KOLLIDON ratio at 2:1, as disclosed in U.S. Pat. No. 6,696,085 patent) exceeds 0.80%. Furthermore, in vitro disintegration time of the latter exceeds 15 seconds.

Increasing EUDRAGIT: KOLLIDON ratio up to 1:3 (Example 26) allows further shortening of in vitro disintegration time (inferior to 10 seconds) while keeping friability at acceptable levels (less than 0.80%)

Example 27, presenting EUDRAGIT and KOLLIDON in typical proportions as disclosed in the U.S. Pat. No. 6,696,085, fails having friability complying with USP requirements (>1%), albeit presenting a very short in vitro disintegration time.

In light of Examples 20 to 27, it can be affirmed that a combination of EUDRAGIT and KOLLIDON, EUDRAGIT and KOLLIDON being present up to 15.0%, at ratios ranging from 1:1 to 1:3, allows manufacture of orally disintegrating tablets having an in vitro disintegration time that is less than 15 seconds, while having a friability not higher than 0.8% according to the U.S. Pharmacopoeia test.

### Examples 28, 29, 30 and 31

Examples recited so far relate to tablets with a mass of 300 mg or less. Formulations of the present invention with a tablet mass of 500 mg were also investigated.

Tablets were produced by direct compression on the same alternative tableting machine (KORSCH EK0) as in the U.S. Pat. No. 6,696,085, following the “EUDRAGIT” formula indicated in Table 24 herein.

Tablets were produced targeting USP friability not higher than 1%.

The tablets are of a flat, bevel-edged shape. Diameter is 13 mm. Mass is about 500 mg.

TABLE 24

| | Content (% wt) | | | |
|---|---|---|---|---|
| | Example | Example | Example | Example |
| Components | 28 | 29 | 30 | 31 |
| EUDRAGIT L 100-55 ® | — | 10.0 | — | 10.0 |
| (Rohm) | | | | |
| KOLLIDON CL ® | — | — | 10.0 | 10.0 |
| (BASF) | | | | |
| Mannitol PEARLITOL | 98.0 | 88.0 | 88.0 | 78.0 |
| 200SD (Roquette) | | | | |
| Mint flavor (Givaudan) | 0.50 | 0.50 | 0.50 | 0.50 |
| Sweetener SUCRAM | 0.50 | 0.50 | 0.50 | 0.50 |
| PH821 ® (Pancosma) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. Results are set out in Table 25 herein.

TABLE 25

| | | In vitro | USP |
|---|---|---|---|
| | Hardness | disintegration time | friability |
| Formula | (N) | (sec) | (%) |
| Example 28 | 33.1 | About 4 minutes | Failed |
| Example 29 | 35.2 | 1 minute 28 sec | 1.04 |
| Example 30 | 31.0 | 22 | Failed |
| Example 31 | 31.0 | 15 | 0.96 |

As observed in Examples 4, 5 and 6 on tablets having a diameter of 7 mm and a mass of 100 mg, and in Examples 20, 21, 22 and 23 on tablets having a diameter of 11 mm and a mass of 300 mg, addition of type-C polymethacrylate (Example 29) to a formulation containing only mannitol (reference, Example 28) allows decreasing significantly the time for in vitro disintegration while improving significantly friability.

Noteworthy, further addition of crospovidone (so that type-C polymethacrylate and crospovidone are present in a 1:1 ratio, Example 31) allows for an even faster in vitro disintegration (less than 20 seconds, at 15 seconds) while obtaining tablets passing the USP friability test.

Noteworthy, crospovidone as the only disintegrant (Example 30) did not enable to obtain tablets disintegrating within less than 15 seconds in vitro.

Outstandingly, friability of the Example 31 (0.96%) is also better than the friability of the control, i.e. not containing the type-C Polymethacrylate.

### Examples 32, 33, 34 and 35

Formulations of the present invention with a tablet mass of 500 mg varying the ratio of the two disintegrants/co-disintegrants were investigated.

Tablets were produced by direct compression on the same alternative tableting machine (KORSCH EK0) as in the U.S. Pat. No. 6,696,085, following the “EUDRAGIT” formula indicated in Table 26 herein.

Tablets were produced targeting USP friability not higher than 1%.

The tablets are of a flat, bevel-edged shape. Diameter is 13 mm. Mass is about 500 mg.

TABLE 26

| | Content (% wt) | | | |
|---|---|---|---|---|
| | Example | Example | Example | Example |
| Components | 32 | 33 | 34 | 35 |
| EUDRAGIT L 100-55 ® | 5.00 | 10.0 | 5.00 | 30.0 |
| (Rohm) | | | | |
| KOLLIDON CL ® | 10.0 | 5.00 | 15.0 | 15.0 |
| (BASF) | | | | |
| Mannitol PEARLITOL | 83.0 | 83.0 | 78.0 | 53.0 |
| 200SD (Roquette) | | | | |
| Mint flavor (Givaudan) | 0.50 | 0.50 | 0.50 | 0.50 |
| Sweetener SUCRAM | 0.50 | 0.50 | 0.50 | 0.50 |
| PH821 ® (Pancosma) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. Results are set out in Table 27 herein.

TABLE 27

| | Hardness | In vitro disintegration time | USP friability |
|---|---|---|---|
| Formula | (N) | (sec) | (%) |
| Example 32 | 31.7 | 16.6 | 0.82 |
| Example 33 | 31.8 | 21 | Failed |
| Example 34 | 30.9 | 15 | 0.95 |
| Example 35 | 30.7 | 14 | 1.58 |

Example 32 (EUDRAGIT: KOLLIDON ratio at 1:2) presents a friability of 0.82%, while friability of Example 33 (EUDRAGIT: KOLLIDON ratio at 2:1, as disclosed in U.S. Pat. No. 6,696,085 patent) exceeds 1.00%. Furthermore, in vitro disintegration time of the latter exceeds 20 seconds.

Increasing EUDRAGIT: KOLLIDON ratio up to 1:3 (Example 34) allows further shortening of in vitro disintegration time while keeping friability at acceptable levels (less than 1.00%)

Example 35, presenting EUDRAGIT and KOLLIDON in typical proportions as disclosed in the U.S. Pat. No. 6,696,085, fails having friability complying with USP requirements (>1%), albeit presenting a very short in vitro disintegration time.

In light of Examples 28 to 35, it can be affirmed that a combination of EUDRAGIT and KOLLIDON, EUDRAGIT and KOLLIDON being present up to 15.0%, at ratios ranging from 1:1 to 1:3, allows manufacture of orally disintegrating tablets having an in vitro disintegration time that is less than 20 seconds, while having a friability not higher than 1.0% according to the U.S. Pharmacopoeia test.

### Examples 36, 37, 38, and 39

Examples disclosed in the U.S. Pat. No. 6,696,085 patent are typically comprising EUDRAGIT 30.0% wt and KOLLIDON 15.0% wt, and dextrose (ROFEROSE® G, Roquette), a sugar, as the main diluent. Furthermore, as already highlighted in Example 1 of the present invention, Examples 2, 3, 4 and 5 of U.S. Pat. No. 6,696,085 contain a small amount of sorbitol (about 3.00% by weight of the total tablet) in order to “improve the compressibility of the mixture ofpowders”. Addition of sorbitol consequently results in the possibility to “work with lower hardnesses while obtaining a tablet which is not very friable”.

Formulations as recited in the U.S. Pat. No. 6,696,085 patent were compared with formulations of the present invention.

Tablets were produced by direct compression on the same alternative tableting machine (KORSCH EK0) as in the U.S. Pat. No. 6,696,085, following the “EUDRAGIT” formula indicated in Table 28 herein.

Tablets were produced targeting USP friability not higher than 1%.

The tablets are of a flat, bevel-edged shape. Diameter is 11 mm. Mass is about 300 mg.

TABLE 28

| | Content (% wt) | | |
|---|---|---|---|
| | Example | Example | Example |
| Components | 36 | 37 | 38 |
| EUDRAGIT L 100-55 ® (Röhm) | 30.0 | 10.0 | 10.0 |
| KOLLIDON CL ® (BASF) | 15.0 | 10.0 | 10.0 |
| Dextrose Roferose G (Roquette) | 50.0 | 75.0 | — |
| Sorbitol P100T (Roquette) | 3.00 | 3.00 | — |
| Mannitol PEARLITOL 200SD | — | — | 78.0 |
| (Roquette) | | | |
| Mint flavor (Givaudan) | 0.50 | 0.50 | 0.50 |
| Sweetener SUCRAM | 0.50 | 0.50 | 0.50 |
| PH821 ® (Pancosma) | | | |
| Magnesium stearate | 1.00 | 1.00 | 1.00 |

The pharmacotechnical parameters, and in particular the average hardness, the average friability and the average in vitro disintegration time for these tablets were then measured. Results are set out in Table 29 herein.

TABLE 29

| | Hardness | In vitro disintegration time | USP friability |
|---|---|---|---|
| Formula | (N) | (sec) | (%) |
| Example 36 | 19.0 | 8.9 | 3.94 |
| Example 37 | 21.5 | 8.7 | Failed |
| Example 38 | 20.5 | 11.4 | 0.79 |

Comparing Example 36 with Example 37 clearly demonstrates the positive effect EUDRAGIT has on friability. However, none of these two examples illustrating the U.S. Pat. No. 6,696,085 patent presents friability lower than 1.0% according to the US Pharmacopoeia test.

Surprisingly, replacing total dextrose (and sorbitol) by mannitol (Example 38) allows obtaining orally disintegrating tablets having in vitro disintegration time inferior to 15 seconds while at the same time passing the USP friability test.

To further demonstrate the additional benefits of the present invention over the invention recited in the U.S. Pat. No. 6,696,085, Example 39 (tablet mass of 100 mg) was compared with “Reference Example 2” and “EUDRAGIT formula Example 2”, respectively, disclosed in the U.S. Pat. No. 6,696,085 patent (Table 30 herein).

TABLE 30

| | Content (% wt) | | |
|---|---|---|---|
| | Reference | EUDRAGIT | |
| | Example 2 | formula Example | |
| Components | (6,696,085) | 2 (6,696,085) | Example 39 |
| EUDRAGIT L 100-55 ® | — | 30.0 | 10.0 |
| (Röhm) | | | |
| KOLLIDON CL ® | 15.0 | 15.0 | 10.0 |
| (BASF) | | | |
| Dextrose Roferose G | 78.75 | 48.75 | — |
| (Roquette) | | | |
| Sorbitol P100T | 3.00 | 3.00 | — |
| (Roquette) | | | |
| Mannitol PEARLITOL | — | — | 79.0 |
| 200SD (Roquette) | | | |
| Flavor (Givaudan) | | | |
| Sweetener SUCRAM | q.s. 100.0 | q.s. 100.0 | q.s. 100.0 |
| PH821 ® (Pancosma) | | | |
| Magnesium stearate | | | |

The pharmacotechnical parameters, and in particular the average hardness, the average friability and the average in vitro disintegration time for these tablets were then measured. Results are set out in Table 31 herein.

TABLE 31

| | | In vitro | USP |
|---|---|---|---|
| | Hardness | disintegration | friability |
| Formula | (N) | time (sec) | (%) |
| Reference Example 2 | 10.8 | 13 | Not available |
| (6,696,085) | | | |
| EUDRAGIT formula | 3.9 | 7 | Not available |
| Example 2 (6,696,085) | | | |
| Example 39 | 22.9 | 9.8 | 0.55 |

Both compositions of the U.S. Pat. No. 6,696,085 patent resulted in soft tablets whose friability could not even be determined.

Conversely, formulation of the present invention (Example 39) allows obtaining orally disintegrating tablets having in vitro disintegration time inferior to 10 seconds while at the same time passing the USP friability test.

### Examples 40, 41, 42 and 43

Examples recited so far relate to tablets with a mass of 500 mg or less. Formulations of the present invention with a tablet mass of 900 mg were also investigated.

Tablets were produced by direct compression on the same alternative tableting machine (KORSCH EK0) than in the U.S. Pat. No. 6,696,085, following the “EUDRAGIT” formula indicated in Table 32 herein.

Tablets were produced targeting USP friability not higher than 1%.

The tablets are of a flat, bevel-edged shape. Diameter is 15 mm. Mass is about 850-900 mg.

TABLE 32

| | Content (% wt) | | | |
|---|---|---|---|---|
| | Example | Example | Example | Example |
| Components | 40 | 41 | 42 | 43 |
| EUDRAGIT L 100-55 ® | — | 10.0 | — | 10.0 |
| (Röhm) | | | | |
| KOLLIDON CL ® | — | — | 10.0 | 10.0 |
| (BASF) | | | | |
| Mannitol PEARLITOL | 98.0 | 88.0 | 88.0 | 78.0 |
| 200SD (Roquette) | | | | |
| Mint flavor (Givaudan) | 0.50 | 0.50 | 0.50 | 0.50 |
| Sweetener SUCRAM | 0.50 | 0.50 | 0.50 | 0.50 |
| PH821 ® (Pancosma) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. Results are set out in Table 33 herein.

TABLE 33

| | Hardness | In vitro disintegration time | USP friability |
|---|---|---|---|
| Formula | (N) | (sec) | (%) |
| Example 40 | 49.9 | 450 | 1.54 |
| | | (7 min 30) | |
| Example 41 | 51.4 | 180 | 1.10 |
| | | (3 min) | |
| Example 42 | 52.0 | 28 | 2.05 |
| Example 43 | 49.3 | 20 | 0.97 |

As observed in Examples 4, 5 and 6 on tablets having a diameter of 7 mm and a mass of 100 mg, in Examples 20, 21, 22 and 23 on tablets having a diameter of 11 mm and a mass of 300 mg, and in Examples 28, 29, 30 and 31 on tablets having a diameter of 13 mm and a mass of 500 mg, addition of type-C polymethacrylate (Example 41) to a formulation containing only mannitol (reference, Example 40) allows decreasing significantly the time for in vitro disintegration while improving friability.

Noteworthy, further addition of crospovidone (so that type-C polymethacrylate and crospovidone are present in a 1:1 ratio, Example 43) allows for an even faster in vitro disintegration (less than 30 seconds, at 20 seconds) while obtaining tablets passing the USP friability test.

Outstandingly, friability and in vitro disintegration time of the Example 43 (0.96%, respectively 20 seconds) are lower than those obtained for the control, i.e. not containing the type-C Polymethacrylate, or those obtained for the two disintegrants separately.

### Examples 44, 45, 46 and 47

Formulations of the present invention with a tablet mass of 900 mg making varying the ratio of the two disintegrants/co-disintegrants were investigated.

Tablets were produced by direct compression on the same alternative tableting machine (KORSCH EK0) as in the U.S. Pat. No. 6,696,085, following the “EUDRAGIT” formula indicated in Table 34 herein.

Tablets were produced targeting USP friability not higher than 1%.

The tablets are of a flat, bevel-edged shape. Diameter is 15 mm. Mass is about 850-900 mg.

TABLE 34

| | Content (% wt) | | | |
|---|---|---|---|---|
| | Example | Example | Example | Example |
| Components | 44 | 45 | 46 | 47 |
| EUDRAGIT L 100-55 ® | 5.00 | 10.0 | 5.00 | 30.0 |
| (Röhm) | | | | |
| KOLLIDON CL ® | 10.0 | 5.00 | 15.0 | 15.0 |
| (BASF) | | | | |
| Mannitol PEARLITOL | 83.0 | 83.0 | 78.0 | 53.0 |
| 200SD (Roquette) | | | | |
| Mint flavor (Givaudan) | 0.50 | 0.50 | 0.50 | 0.50 |
| Sweetener SUCRAM | 0.50 | 0.50 | 0.50 | 0.50 |
| PH821 ® (Pancosma) | | | | |
| Magnesium stearate | 1.00 | 1.00 | 1.00 | 1.00 |

The pharmacotechnical parameters, and in particular the average hardness, the average friability and the average in vitro disintegration time for these tablets were then measured. Results are set out in Table 35 herein.

TABLE 35

| | Hardness | In vitro disintegration time | USP friability |
|---|---|---|---|
| Formula | (N) | (sec) | (%) |
| Example 44 | 50.1 | 24 | 0.88 |
| Example 45 | 51.8 | 31 | 0.74 |
| Example 46 | 50.7 | 23 | 0.77 |
| Example 47 | 50.0 | 14 | 0.42 |

Example 44 (EUDRAGIT: KOLLIDON ratio at 1:2) presents an vitro disintegration time of 24 seconds, while an vitro disintegration time of Example 45 (EUDRAGIT: KOLLIDON ratio at 2:1, as disclosed in U.S. Pat. No. 6,696,085 patent) exceeds 30 seconds.

Increasing EUDRAGIT: KOLLIDON ratio up to 1:3 (Example 46) allows further improvement of friability while keeping in vitro disintegration time at acceptable levels (less than 30 seconds).

Example 47, presenting EUDRAGIT and KOLLIDON in typical proportions as disclosed in the U.S. Pat. No. 6,696,085, fails being manufactured under similar conditions since mass of the tablet could not exceed 750 mg, because of the greater density of the bulk caused by the presence of large quantities of EUDRAGIT and KOLLIDON (thereby witnessing that these two disintegrants, and especially EUDRAGIT, are significantly increasing the porosity of the tablet, which is a necessary pre-requisite for fast disintegration). Hence direct comparison with Examples 44, 45 and 46 is not possible. Furthermore, the large amount of the disintegrants in this formulation (especially EUDRAGIT, present at about 225 mg) is responsible for a “soapy”, unpleasant taste. Therefore use of such large amounts of disintegrants is not recommended.

In light of Examples 40 to 47, it can be affirmed that combination of EUDRAGIT and KOLLIDON, EUDRAGIT and KOLLIDON being present up to 15.0%, at ratios ranging from 1:1 to 1:3, allows manufacture of orally disintegrating tablets having an in vitro disintegration time that is less than 30 seconds, while having a friability not higher than 1.0% according to the U.S. Pharmacopoeia test.

### Example 48, 49, 50 and 51

Examples recited so far relate to tablets containing mannitol having an average particle size of about 200 microns (PEARLITOL 200SD, ROQUETTE). Formulations of the present invention with mannitol having an average particle size of about 100 microns (PEARLITOL 100SD, ROQUETTE) were also investigated.

Tablets were produced by direct compression on the same alternative tableting machine (KORSCH EK0) than in the U.S. Pat. No. 6,696,085, following the “EUDRAGIT” formula indicated in Table 36 herein.

Tablets were produced targeting USP friability not higher than 1%.

The tablets are of a flat, bevel-edged shape. Diameter is 11 mm. Mass is about 300 mg.

TABLE 36

| | Content (% wt) | | | |
|---|---|---|---|---|
| | Example | Example | Example | Example |
| Components | 48 | 49 | 50 | 51 |
| EUDRAGIT L 100-55 ® | — | 10.0 | — | 10.0 |
| (Röhm) | | | | |
| KOLLIDON CL ® | — | — | 10.0 | 10.0 |
| (BASF) | | | | |
| Mannitol PEARLITOL | 97.73 | 87.73 | 87.73 | 77.73 |
| 100SD (Roquette) | | | | |
| Anis flavor (Firmenich) | 0.47 | 0.47 | 0.47 | 0.47 |
| Sweetener SUCRAM | 0.33 | 0.33 | 0.33 | 0.33 |
| PH821 ® (Pancosma) | | | | |
| Magnesium stearate | 1.47 | 1.47 | 1.47 | 1.47 |

The pharmacotechnical parameters, and in particular the average hardness, the average friability and the average in vitro disintegration time for these tablets were then measured. Results are set out in Table 37 herein.

TABLE 37

| | Hardness | In vitro disintegration time | USP friability |
|---|---|---|---|
| Formula | (N) | (sec) | (%) |
| Example 48 | 21.2 | 20 | 1.24 |
| Example 49 | 22.3 | 18 | 0.96 |
| Example 50 | 19.0 | 14 | 1.65 |
| Example 51 | 20.9 | 12 | 1.00 |

As observed in all aforementioned previous Examples 20, 21, 22 and 23, addition of type-C polymethacrylate (Example 49) to a formulation containing only mannitol (reference, Example 48) allows decreasing the time for in vitro disintegration while improving friability. Crospovidone as the only disintegrant (Example 50) allows for significantly shortening in vitro disintegration time (less than 15 seconds), but also for worsening friability.

Outstandingly, combined addition of type-C polymethacrylate and crospovidone so that type-C polymethacrylate and crospovidone are present in a 1:1 ratio, Example 51) allows for an even faster in vitro disintegration (12 seconds) while maintaining a very low friability (less than 1.00% according to the USP friability test).

### Example 52, 53, 54 and 55

Formulations of the present invention with a mannitol having an average particle size of about 100 microns (PEARLITOL 100SD, ROQUETTE) making varying the ratio of the two disintegrants/co-disintegrants were investigated.

Tablets were produced by direct compression on the same alternative tableting machine (KORSCH EK0) than in the U.S. Pat. No. 6,696,085, following the “EUDRAGIT” formula indicated in Table 38 herein.

Tablets were produced targeting USP friability not higher than 0.80%.

The tablets are of a flat, bevel-edged shape. Diameter is 11 mm. Mass is about 300 mg.

TABLE 38

| | Content (% wt) | | | |
|---|---|---|---|---|
| | Example | Example | Example | Example |
| Components | 52 | 53 | 54 | 55 |
| EUDRAGIT L 100-55 ® | 5.00 | 10.0 | 5.00 | 30.0 |
| (Röhm) | | | | |
| KOLLIDON CL ® | 10.0 | 5.00 | 15.0 | 15.0 |
| (BASF) | | | | |
| Mannitol PEARLITOL | 82.73 | 82.73 | 72.73 | 52.73 |
| 100SD (Roquette) | | | | |
| Anis flavor (Firmenich) | 0.47 | 0.47 | 0.47 | 0.47 |
| Sweetener SUCRAM | 0.33 | 0.33 | 0.33 | 0.33 |
| PH821 ® (Pancosma) | | | | |
| Magnesium stearate | 1.47 | 1.47 | 1.47 | 1.47 |

The pharmacotechnical parameters, and in particular the average hardness, the average friability and the average in vitro disintegration time for these tablets were then measured. Results are set out in Table 39 herein.

TABLE 39

| | Hardness | In vitro disintegration time | USP friability |
|---|---|---|---|
| Formula | (N) | (sec) | (%) |
| Example 52 | 21.0 | 13 | 0.95 |
| Example 53 | 21.8 | 16 | 0.83 |
| Example 54 | 20.0 | 10.6 | 0.78 |
| Example 55 | 20.0 | 10.3 | 1.15 |

Example 52 (EUDRAGIT: KOLLIDON ratio at 1:2) presents an vitro disintegration time of 13 seconds, while an vitro disintegration time of Example 53 (EUDRAGIT: KOLLIDON ratio at 2:1, as disclosed in U.S. Pat. No. 6,696,085 patent) exceeds 15 seconds. However, friability exceeds 0.80% according to the USP/NF friability test.

Increasing EUDRAGIT: KOLLIDON ratio up to 1:3 (Example 54) allows obtaining tablets passing USP/NF friability test while keeping in vitro disintegration time at acceptable levels (less than 15 seconds)

Example 55, presenting EUDRAGIT and KOLLIDON in typical proportions as disclosed in the U.S. Pat. No. 6,696,085, fails having friability complying with USP requirements (>1%), albeit presenting a very short in vitro disintegration time.

In light of Examples 48 to 55, it can be affirmed that various types of mannitol intended for direct compression may be used in combination with EUDRAGIT and KOLLIDON, EUDRAGIT and KOLLIDON being present up to 15.0%, at ratios ranging from 1:1 to 1:3, to allow manufacture of orally disintegrating tablets having an in vitro disintegration time that is less than 30 seconds, while having a friability not higher than 1.0% according to the U.S. Pharmacopoeia test.

### Example 56 and 57

All Examples of the present invention presented so far contain crospovidone as the second disintegration agent. Example with hypromellose as the second disintegration agent, a disintegrant widely known in the related art of oral solid dosage forms, was investigated.

Tablets were produced by direct compression on the same alternative tableting machine (KORSCH EK0) than in the U.S. Pat. No. 6,696,085, following the “EUDRAGIT” formula indicated in Table 40 herein.

Tablets were produced targeting USP friability not higher than 0.80%.

The tablets are of a flat, bevel-edged shape. Diameter is 7 mm. Mass is about 100 mg.

TABLE 40

| | Content (% wt) | |
|---|---|---|
| Components | Example 56 | Example 57 |
| EUDRAGIT L 100-55 ® (Röhm) | 5.00 | 5.00 |
| KOLLIDON CL ® (BASF) | 10.0 | — |
| Methocel K100M ® (COLORCON) | — | 10.0 |
| Mannitol PEARLITOL 200SD (Roquette) | 84.0 | 84.0 |
| Magnesium stearate | 1.00 | 1.00 |

The pharmacotechnical parameters, and in particular the average hardness, the average friability and the average in vitro disintegration time for these tablets were then measured. Results are set out in Table 41 herein.

TABLE 41

| | Hardness | In vitro disintegration time | USP friability |
|---|---|---|---|
| Formula | (N) | (sec) | (%) |
| Example 56 | 31.1 | 9.66 | 0.30 |
| Example 57 | 29.6 | More than 2 minutes | 0.42 |

Both crospovidone and hypromellose allows for obtaining oral tablets having a very low friability, low enough to comply with the USP/NF requirement for friability (less than 0.80%).

However, the type-C polymethacrylate:crospovidone combination shows unexpected benefit over a type-C polymethacrylate:hypromellose combination in regards of in vitro disintegration time: besides having an unacceptably long disintegration time—longer than 2 minutes (sic)—, hypromellose is also associated with the formation of a very thick gel which slowly swells when in contact with the saliva, being responsible for a very unpleasant palatability and mouth feel. This is also true, to a more or less similar extent, with other cellulose derivatives. Crospovidone being a wicking agent rather than a swelling agent does not present such a disadvantage.

### Examples 58 and 59

All examples of the present invention presented herein so far contained direct compression mannitol (PEARLITOL 100SD or PEARLITOL 200SD, ROQUETTE). Substitution of direct-compression grade by non direct-compression grade of mannitol was herein investigated.

Tablets were produced by direct compression on the same alternative tableting machine (KORSCH EK0) than in the U.S. Pat. No. 6,696,085, following the “EUDRAGIT” formula indicated in Table 42 herein. Tablets were produced targeting USP friability not higher than 0.80%. The tablets are of a flat, bevel-edged shape. Diameter is 7 mm. Mass is about 115 mg.

TABLE 42

| | Content (% wt) | |
|---|---|---|
| Components | Example 58 | Example 59 |
| Meloxicam | 6.52 | 6.52 |
| EUDRAGIT L 100-55 ® (Röhm) | 5.22 | 5.22 |
| KOLLIDON CL ® (BASF) | 10.43 | 10.43 |
| Mannitol PEARLITOL 200SD (Roquette) | 72.96 | — |
| Mannitol PEARLITOL 160C (Roquette) | — | 72.96 |
| Lemon flavor | 2.17 | 2.17 |
| Flavor enhancer | 0.87 | 0.87 |
| Sweetener | 0.87 | 0.87 |
| Magnesium stearate | 1.00 | 1.00 |

PEARLITOL 200SD is a spray-dried directly-compressible grade of mannitol specifically intended for chewable tablets and orally disintegrating tablets: average particle size is about 200 microns (0.5% of particles larger than 315 microns, 47% larger than 150 microns, 44% larger than 75 microns, and 9% smaller than 75 microns). PEARLITOL 160C is a standard powdered crystallized mannitol grade specifically for wet granulation applications; average particle size is about 160 microns. The pharmacotechnical parameters, and in particular the average hardness, the average friability and the average in vitro disintegration time for these tablets were then measured. Results are set out in Table 43 herein.

TABLE 43

| | Hardness | In vitro disintegration time | USP friability |
|---|---|---|---|
| Formula | (N) | (sec) | (%) |
| Example 58 | 25 | 8.2 | 0.18 |
| Example 59 | NMT 17 | N/A | FAILURE |

Capping at very low compression forces prevented tablets of Example 59 to be compressed at hardness sufficient to allow the friability being lower than 1.00% according to USP/NF. Tablets exhibiting the highest hardness achievable with this composition (17 Newtons) did not even pass the friability test according to USP/NF (several tablets broke during the test as well as during its repetition).

On the contrary, tablets of the Example 58 were within expected specifications, thereby demonstrating the usefulness of direct-compression grade of mannitol in the formulations of the present invention.

### Example 60

Formulations of the present invention with a tablet mass of 300 mg comparing the suppliers of type C Polymethacrylate were investigated. These formulations appear in Table 44 herein.

Tablets were produced by direct compression targeting USP friability not higher than 1%.

TABLE 44

| | Content (% wt) | |
|---|---|---|
| Components | Example 24 | Example 60 |
| EUDRAGIT L 100-55 ® (Röhm) | 5.00 | / |
| Kollicoat MAE 100P (BASF) | / | 5.00 |
| KOLLIDON CL ® (BASF) | 10.0 | 10.0 |
| Mannitol PEARLITOL 200SD (Roquette) | 83.0 | 83.0 |
| Mint flavor (Givaudan) | 0.50 | 0.50 |
| Sweetener SUCRAM PH821 ® (Pancosma) | 0.50 | 0.50 |
| Magnesium stearate | 1.00 | 1.00 |

The pharmacotechnical parameters, and in particular the average hardness, the average friability and the average in vitro disintegration time for these tablets were then measured. Results are set out in Table 45 herein.

TABLE 45

| | | In | USP |
|---|---|---|---|
| Formula | Hardness (N) | vitro disintegration time (sec) | friability (%) |
| Example 24 | 21.5 | 10.7 | 0.60 |
| Example 60 | 21.9 | 12.0 | 0.56 |

Example 60 exhibits same results as example 24. In vitro disintegration time and friability of example 24 containing type-C polymethacrylate from Röhm and Example 60 containing type-C polymethacrylate from BASF are similar and interchangeable.

### Example 61

A formulation of the present invention containing a combination of norethindrone acetate and ethinyl estradiol. Formula is indicated in Table 46 herein. Diameter is 6 mm. Mass is about 60 mg.

TABLE 46

| Components | Content (mg) | |
|---|---|---|
| Norethindrone acetate | 1.00 | |
| Ethinyl estradiol | 0.02 to 0.04 | |
| Polymethacrylate - EUDRAGIT L 100-55 | 4.00 | |
| Crospovidone - KOLLIDON CL | 8.00 | |
| Lemon flavor | 1.00 | |
| Citric acid anhydrous | 1.00 | |
| Aspartame | 0.50 | |
| Magnesium stearate | 0.60 | |
| Mannitol - PEARLITOL 100SD | qs 60 | mg |

### Example 62

A formulation of the present invention containing Domperidone. Formula is indicated in Table 47 herein. Diameter is 7 mm. Mass is about 100 mg.

TABLE 47

| Components | Content (mg) | |
|---|---|---|
| Domperidone | 10.0 | |
| Polymethacrylate - EUDRAGIT L 100-55 | 6.00 | |
| Crospovidone - KOLLIDON CL | 12.00 | |
| Mint flavor | 2.00 | |
| Acesulfame potassium | 0.50 | |
| Magnesium stearate | 0.50 | |
| Talc | 0.50 | |
| Mannitol - PEARLITOL 200SD | qs 100 | mg |

### Example 63

A formulation of the present invention containing a combination of buprenorphine and naloxone. Formula is indicated in Table 48 herein. Diameter is 11 mm. Mass is about 400 mg.

TABLE 48

| Components | Content (mg) | |
|---|---|---|
| Buprenorphine | 16.0 | |
| Naloxone | 4.00 | |
| Polymethacrylate - EUDRAGIT L 100-55 | 20.0 | |
| Crospovidone - KOLLIDON CL | 30.0 | |
| Orange flavor | 10.0 | |
| Aspartame | 2.00 | |
| Colorant orange FD&C #6 | 0.40 | |
| Magnesium stearate | 4.00 | |
| Mannitol - PEARLITOL 400SD | qs 400 | mg |

### Example 64

A formulation of the present invention containing a combination of Glimepiride and Rosiglitazone. Formula is indicated in Table 49 herein. Diameter is 9 mm. Mass is about 200 mg.

TABLE 49

| Components | Content (mg) | |
|---|---|---|
| Glimepiride | 1.00 to 4.00 | |
| Rosiglitazone | 4.00 | |
| Polymethacrylate - EUDRAGIT L 100-55 | 12.0 | |
| Crospovidone - KOLLIDON CL | 30.0 | |
| Cinnamon flavor | 5.00 | |
| Sweetener | 1.50 | |
| Talc | 3.00 | |
| Magnesium stearate | 1.00 | |
| Mannitol - PEARLITOL 200SD | qs 200 | mg |

### Example 65

A formulation of the present invention containing cetirizine. Formula is indicated in Table 50 herein. Diameter is 8 mm. Mass is about 115 mg.

TABLE 50

| Components | Content (mg) | |
|---|---|---|
| Cetirizine | 10.0 | |
| Polymethacrylate - EUDRAGIT L 100-55 | 10.0 | |
| Crospovidone - KOLLIDON CL | 20.0 | |
| Strawberry flavor | 5.00 | |
| Sweetener | 2.00 | |
| Magnesium stearate | 1.80 | |
| Mannitol - PEARLITOL 100SD | qs 115 | mg |

### Example 66

A formulation of the present invention containing zolpidem. Formula is indicated in Table 51 herein. Diameter is 7 mm. Mass is about 100 mg.

TABLE 51

| Components | Content (mg) | |
|---|---|---|
| Zolpidem | 10.0 | |
| Polymethacrylate - EUDRAGIT L 100-55 | 5.00 | |
| Crospovidone - KOLLIDON CL | 10.0 | |
| Anis flavor | 4.00 | |
| Sweetener | 1.50 | |
| Magnesium stearate | 1.00 | |
| Mannitol - PEARLITOL 200SD | qs 100 | mg |

### Example 67

A formulation of the present invention containing atorvastatin. Formula is indicated in Table 52 herein. Diameter is 13 mm. Mass is about 600 mg.

TABLE 52

| Components | Content (mg) | |
|---|---|---|
| Atorvastatin | 40.0 | |
| Polymethacrylate - EUDRAGIT L 100-55 | 30.0 | |
| Crospovidone - KOLLIDON CL | 60.0 | |
| Vanilla flavor | 9.00 | |
| Sweetener | 4.50 | |
| Magnesium stearate | 8.00 | |
| Mannitol - PEARLITOL 400SD | qs 600 | mg |

### Example 68

A formulation of the present invention containing candesartan. Formula is indicated in Table 53 herein. Diameter is 7 mm. Mass is about 130 mg.

TABLE 53

| Components | Content (mg) | |
|---|---|---|
| Candesartan | 8.00 | |
| Polymethacrylate - EUDRAGIT L 100-55 | 8.00 | |
| Crospovidone - KOLLIDON CL | 14.0 | |
| Flavor | 3.40 | |
| Glyceryl behenate | 2.60 | |
| Mannitol - PEARLITOL 200SD | qs 130 | mg |

### Example 69

A formulation of the present invention containing sildenafil. Formula is indicated in Table 54 herein. Diameter is 11 mm. Mass is about 300 mg.

TABLE 54

| Components | Content (mg) | |
|---|---|---|
| Sildenafil | 50.0 | |
| Polymethacrylate - EUDRAGIT L 100-55 | 30.0 | |
| Crospovidone - KOLLIDON CL | 30.0 | |
| Lime flavor | 6.00 | |
| Citric acid | 6.00 | |
| Sodium stearyl fumarate | 1.50 | |
| Mannitol - PEARLITOL 200SD | qs 300 | mg |

### Example 70

A formulation of the present invention containing amlodipine. Formula is indicated in Table 55 herein. Diameter is 8 mm. Mass is about 150 mg.

TABLE 55

| Components | Content (mg) | |
|---|---|---|
| Amlodipine | 5.00 | |
| Polymethacrylate - EUDRAGIT L 100-55 | 6.00 | |
| Crospovidone - KOLLIDON CL | 15.0 | |
| Peppermint flavor | 5.00 | |
| Aspartame | 1.50 | |
| Sodium stearyl fumarate | 1.50 | |
| Mannitol - PEARLITOL 200SD | qs 150 | mg |

### Example 71

A formulation of the present invention containing zafirlukast. Formula is indicated in Table 56 herein. Diameter is 9 mm. Mass is about 220 mg.

TABLE 56

| Components | Content (mg) | |
|---|---|---|
| Zafirlukast | 20.0 | |
| Polymethacrylate - EUDRAGIT L 100-55 | 20.0 | |
| Crospovidone - KOLLIDON CL | 20.0 | |
| Lemon flavor | 4.50 | |
| Citric acid | 3.00 | |
| Aspartame | 2.20 | |
| Sodium stearyl fumarate | 1.50 | |
| Mannitol - PEARLITOL 200SD | qs 220 | mg |

Accordingly, the foregoing examples demonstrate that:
1. Orally disintegrating dosage forms as disclosed in U.S. Pat. No. 6,696,085 may possibly contain diluent-type excipients that may promote tooth decay and/or that may be responsible for digestion intolerances and/or that may affect glycemia (such as sucrose, dextrose, glucose, lactose, etc. . . .) and do not pass the USP friability test.
2. Replacement of these diluents by diluents which do not present such drawbacks, such as sugar alcohols, in formulations recited by the U.S. Pat. No. 6,696,085, allows improving friability but do not result in oral dosage forms with acceptable disintegration times.
3. Surprisingly, it was found out that orally disintegrating dosage forms containing mannitol as the sole diluent or as the primary diluent present acceptable in vitro disintegration times while passing the USP friability test. Other sugar alcohols were not found suitable.
4. Orally disintegrating dosage forms up to 150 mg containing substantially only mannitol as the diluent and type-C polymethacrylate:crospovidone ratios ranging from 1:1 to 1:3 present in vitro disintegration times less than 10 seconds and friability less than 0.8%.
5. Orally disintegrating dosage forms up to 300 mg containing substantially only mannitol as the diluent and type-C polymethacrylate:crospovidone ratios ranging from 1:1 to 1:3 present in vitro disintegration times less than 15 seconds and friability less than 0.8%.
6. Orally disintegrating dosage forms up to 500 mg containing substantially only mannitol as the diluent and type-C polymethacrylate:crospovidone ratios ranging from 1:1 to 1:3 present in vitro disintegration times less than 20 seconds and friability less than 1.0%.
7. Orally disintegrating dosage forms up to about 1000 mg containing substantially only mannitol as the diluent and type-C polymethacrylate:crospovidone ratios ranging from 1:1 to 1:3 present in vitro disintegration times less than 30 seconds and friability less than 1.0%.
8. Formulations of point 4 do show a physical stability such that packaging in specific containers and particular caution upon handling are not required.
9. Formulations of point 4 can easily be scaled up for industrial production.
10. Besides not containing excipients mentioned in point 1, formulations of point 4 contain not more than about 20% of the total weight of the tablet (weight of the active ingredient(s) being not considered) of insoluble, swelling excipients which impart a chalky, pasty, gritty mouthfeel.

It will be apparent to those skilled in the art that various modifications and variations can be made in the method and composition of the present invention without departing from the spirit or scope of the invention. Thus, it is intended that the present invention include modifications and variations that are within the scope of the appended claims and their equivalents.

What is claimed is:

1. A rapidly disintegrating orally administratable solid formulation comprising a matrix that includes:
at least one first pH-dependent water-soluble disintegration agent that is at least one Type C methacrylic acid copolymer according to the U.S. Pharmacopoeia National Formulary US/NF;
at least one second water-insoluble disintegration agent which is non-swelling in water; and
a non-cariogenic diluent that does not increase glucose blood levels,
wherein the solid dosage form has a mass of about 50 to about 1000 mg, the at least one first disintegration agent is present in the dosage form in an amount not exceeding 15%, with respect to the total weight of the dosage form, the at least second disintegration agent is present in the dosage form in an amount not exceeding 15% with respect to the total weight of the dosage form, and the at least first and second disintegration agents are present in total amounts that provide a weight ratio of about 1:1 to about 1:3, wherein the dosage form provides at least one of an in vitro or in vivo disintegration time that is less than 30 seconds, and has a friability of 1% or less according to the U.S. Pharmacopoeia test.

2. The formulation of claim 1 which further comprises at least one active ingredient.

3. The formulation of claim 1, wherein the non-cariogenic diluent consists substantially of mannitol and is present in an amount of between 25% and 85% by weight of the formulation.

4. The formulation of claim 1, wherein the formulation is substantially free of lactose or fructose responsible for intestinal discomfort in populations suffering from sugar intolerance or which is substantially free of precursors being metabolized in the human body into lactose or fructose.

5. The formulation of claim 1, which further comprises one or more diluents or fillers, sweeteners, binders, flavors and flavor enhancers, buffers, preservatives, antioxidants, lubricants, bioadhesive agents, colorants, flow agents, plasticizers, film forming agents, coating agents, polishing agents, shining agents, or mixtures thereof.

6. The formulation of claim 5, wherein the sweetener is not contraindicated in populations suffering from phenylketonury.

7. The formulation of claim 1, wherein the type-C methacrylic acid copolymer is a compound having the formula:

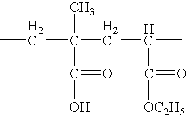

8. The formulation of claim 1, wherein the amount of water-insoluble, non-swelling inactive ingredients in the formulation does not exceed 20% by weight of the total formulation.

9. The formulation of claim 1, wherein the at least one active ingredient is used to treat cardiovascular disorders, respiratory disorders, gastrointestinal disorders, renal disorders, neurologic disorders, psychiatric disorders, endocrinologic disorders, gynecologic and obstetric disorders, urologic disorders, immunologic disorders, bone and joint disorders, disorders of the eyes, ears, nose and throat, dermatologic disorders, hematologic disorders, infectious diseases, oncologic disorders, nutritional disorders.

10. The formulation of claim 1, wherein the at least one active ingredient is selected from the class of hypnotics, analeptics, analgesics, local or general anaesthetics, muscle relaxants, antiepileptics, antiParkinsonian drugs, antiemetics, hormones, anti-hormones, lipid-lowering agents, phosphodiesterase inhibitors, antiarrhythmics, beta-receptor blockers, calcium channel blockers, angiotensin converting enzyme inhibitors, antimigraine agents, antiasthmatic drugs, antitussives drugs, antiacids, modulators of the gastrointestinal motility, antiulcer drugs, laxatives, antidiarrhea drugs, ulcerative colitis and Crohn's disease drugs, choleretic and cholekinetic drugs, diuretics, vitamins, anti-infectives drugs, mitotic inhibitors, cytostatics, narcotics, analgesics, anesthetics.

11. The formulation of claim 1, wherein the at least one active ingredient is selected for the group consisting of meloxicam, androgens, estrogens, progestins, clonazepam, zolpidem, buprenorphine, naloxone, atorvastatin, candesartan, glimepiride, rosiglitazone, domperidone, cetirizine, sildenafil, amlodipine, zafirlukast and combination thereof.

12. The formulation of claim 1, wherein the solid dosage form has a mass of 50 to 150 mg, with the active ingredient being present in the dosage form in an amount not exceeding 15 mg, the at least one first disintegration agent being present in the dosage form in an amount not exceeding 10%, with respect to the total weight of the dosage form, the second disintegration agent being present in the dosage form in an amount not exceeding 10% with respect to the total weight of the dosage form, and with the first and second disintegration agents being present in total amounts that provide a weight ratio of about 1:1 to about 1:2, wherein the dosage form provides at least one of the in vitro or in vivo disintegration time that is less than 10 seconds, and has a friability of 0.8% or less according to the U.S. Pharmacopoeia test when manufactured on rotary tableting machine operated at industrial speeds up to 60 rpm, and being obtained by direct compression.

13. The formulation of claim 1, wherein the solid dosage form has a mass of 150 to 300 mg, with the active ingredient being present in the dosage form in an amount not exceeding 50 mg, the at least one first disintegration agent being present in the dosage form in an amount not exceeding 15%, with respect to the total weight of the dosage form, the second disintegration agent being present in the dosage form in an amount not exceeding 15% with respect to the total weight of the dosage form, and with the first and second disintegration agents being present in total amounts that provide a weight ratio of about 1:1 to about 1:3, wherein the dosage form provides at least one of the in vitro or in vivo disintegration time that is less than 15 seconds, and has a friability of 0.8% or less according to the U.S. Pharmacopoeia test when manufactured on rotary tableting machine operated at industrial speeds up to 60 rpm, and being obtained by direct compression.

14. The formulation of claim 1, wherein the solid dosage form has a mass of 300 to 500 mg, with the active ingredient being present in the dosage form in an amount not exceeding 200 mg, the at least one first disintegration agent being present in the dosage form in an amount not exceeding 15%, with respect to the total weight of the dosage form, the second disintegration agent being present in the dosage form in an amount not exceeding 15% with respect to the total weight of the dosage form, and with the first and second disintegration agents being present in total amounts that provide a weight ratio of about 1:1 to about 1:3, wherein the dosage form provides at least one of the in vitro or in vivo disintegration time that is less than 20 seconds, and has a friability of 1% or less according to the U.S. Pharmacopeia test when manufactured on rotary tableting machine operated at industrial speeds up to 60 rpm, and being obtained by direct compression.

15. The formulation of claim 1, wherein the solid dosage form has a mass of 500 to about 1000 mg, with the active ingredient being present in the dosage form in an amount not exceeding 500 mg, the at least one first disintegration agent being present in the dosage form in an amount not exceeding 15%, with respect to the total weight of the dosage form, the second disintegration agent being present in the dosage form in an amount not exceeding 15% with respect to the total weight of the dosage form, and with the first and second disintegration agents being present in total amounts that provide a weight ratio of about 1:1 to about 1:3, wherein the dosage form provides at least one of the in vitro or in vivo disintegration time that is less than 30 seconds, and has a friability of 1% or less according to the U.S. Pharmacopoeia test when manufactured on rotary tableting machine operated at industrial speeds up to 60 rpm, and being obtained by direct compression.

16. A process for preparing the solid dosage formulation of claim 1, which comprises directly compressing the components to form the formulation.

17. A method of administering an active ingredient which comprises orally administering the formulation of claim 1 to a patient in need thereof so that the solid formulation rapidly disintegrates to administer the active ingredient.
